(19) United States
(12) Patent Application Publication
(10) Pub. No.: US 2025/0339118 A1
(43) Pub. Date: Nov. 6, 2025

(54) Systems and Methods for Detecting Microcalcification Activity

(71) Applicant: Navier Medical Ltd, Perth, Western Australia (AU)

(72) Inventors: Barry Joseph Doyle, Perth, Western Australia (AU); Lachlan James Kelsey, Perth, Western Australia (AU)

(21) Appl. No.: 18/572,997

(22) PCT Filed: Jul. 22, 2022
(86) PCT No.: PCT/AU2022/050779

§ 371 Date: Dec. 21, 2023

(30) Foreign Application Priority Data
Jul. 23, 2021 (AU) 2021902266

(51) Int. Cl. A61B 6/50; A61B 5/00; A61B 5/02; A61B 6/00; A61B 6/03; G06T 7/00; G06T 7/10; G06T 7/62; G16H 30/40

(52) U.S. Cl. CPC A61B 6/504; A61B 5/0035; A61B 5/004; A61B 5/0066; A61B 5/02007; A61B 6/032; A61B 6/037; A61B 6/5217; A61B 6/5247; G06T 7/0012; G06T 7/10; G06T 7/62; G16H 30/40; G06T 2207/10081; G06T 2207/10088; G06T 2207/10101; G06T 2207/10104; G06T 2207/10116; G06T 2207/10132; G06T 2207/20081; G06T 2207/20084; G06T 2207/30048; G06T 2207/30104; G06T 2207/30204

(57) ABSTRACT

Systems and methods of predicting microcalcification activity in a vascular vessel comprising either an artery or a vein, comprising the steps of: (a) measuring patient data comprising one or more of: the existence of and/or quantity of coronary plaques or visible markers of disease in a vascular tissue sample; the existence of and/or quantity of healthy tissue in the vascular tissue sample; one or more features that define an abnormal hemodynamic environment in a vessel; one or more geometric features that are associated with vascular remodeling and which influence hemodynamics in a vessel, and/or one or more material properties that influence vascular hemodynamics; and (b) calculating the microcalcification activity in the vessel as a function of the measurements taken in Step (a).

### FIELD OF THE INVENTION

The present invention relates generally to the field estimating/predicting ¹⁸F—NaF uptake in vascular tissues, and in particular relates to estimating/predicting ¹⁸F—NaF uptake in vascular tissues without using ¹⁸F—NaF PET imaging modalities and will be described hereinafter with reference to this application. However, it will be appreciated that the invention is not limited to this particular field of use.

### BACKGROUND

Any discussion of the background art throughout the specification should in no way be considered as an admission that such background art is prior art, nor that such background art is widely known or forms part of the common general knowledge in the field in Australia or worldwide.

All references, including any patents or patent applications, cited in this specification are hereby incorporated by reference. No admission is made that any reference constitutes prior art. The discussion of the references states what their authors assert, and the applicants reserve the right to challenge the accuracy and pertinence of the cited documents. It will be clearly understood that, although a number of prior art publications are referred to herein, this reference does not constitute an admission that any of these documents forms part of the common general knowledge in the art, in Australia or in any other country.

The present discussion is related specifically to imaging and analysis of microcalcification activity in coronary arteries leading to coronary heart disease, however, the disclosure herein is readily applicable to the patient's vasculature throughout the body, not limited to coronary arteries, and particularly including at least a patient's carotid artery, cerebral artery, aorta, peripheral artery, or any artery or vein in the vascular system.

Coronary heart disease (CHD) is the leading cause of death globally. In 2015 CHD affected 110 M people and resulted in 8.9 M deaths. It makes up 16% of all deaths making it the most common cause of death globally. Coronary heart disease is also the single leading cause of death in Australia (12% of all deaths; 1 person every 27 minutes).

In 2016 the American Heart Association (HCA) reported 15.5 M persons>20 years of age in the USA have CHD. The reported prevalence increases with age for both women and men.

Approximately 25-30% of patients admitted with a heart attack due to CHD will die or be re-admitted at least once with a further clinical-event within 3 years, representing a substantial health burden. Recurrent events occur despite routine use of several proven risk reduction strategies including inpatient coronary angiography and revascularization, statins, dual antiplatelet therapy, p-blockers, ACE-inhibitors, and lifestyle advice. Novel preventative therapies are being assessed, for example targeting inflammatory processes relevant to plaque rupture, but will come at a high cost and may have unfavourable side effects. It is widely acknowledged that there is an unmet need for improved risk stratification to better target these therapies only at those more likely to benefit.

When a patient is suspected of CHD, the treating cardiologist will typically perform Percutaneous Coronary Intervention (PCI). PCI involves the insertion of a catheter into the heart via the wrist or groin. The injection of a contrast dye under x-ray imaging will highlight the blood flow and reveal any narrowing of the arteries. This part of the procedure is called a coronary angiogram and can be recorded for later analysis. Next, the cardiologist will guide another catheter towards the blockage and either open the blockage with a balloon, or place a stent into the blocked region. The PCI procedure helps in providing relief for the symptoms of coronary heart diseases and reduces damage to the heart after or during a heart attack. The global PCI market was USD 10B in 2017 and is expected to reach over USD$15B by 2023.

During the coronary angiogram and prior to angioplasty (balloon treated) or stenting, important information can be obtained as to the pressure difference along a diseased artery due to the blockage that can inform the decision to stent or not. This is called Fractional Flow Reserve (FFR) and measures the pressure differences across a coronary artery stenosis (narrowing, usually due to atherosclerosis) to determine the likelihood that the stenosis impedes oxygen delivery to the heart muscle (myocardial ischemia). FFR has become the standard of care for assessment of the physiological significance of coronary artery disease (CAD). When FFR is used to guide percutaneous coronary intervention (PCI), clinical outcomes are improved, fewer stents are deployed, and costs are reduced.

However, even in countries where FFR is most frequently used, FFR is used in <10% of PCI procedures and far fewer diagnostic cases; hence, despite the advantages, clinical uptake remains extremely low. This is due to a combination of factors related to practicality, time, and cost. Using computational fluid dynamics (CFD) to compute a “virtual” FFR (vFFR) from the coronary angiogram (CAG) offers the benefits of physiologically guided PCI without the drawbacks which limit the invasive technique.

vFFR can be calculated based upon a 3-dimensional (3D) reconstruction of the coronary anatomy from the coronary CT angiogram (image) using CFD modelling. Recently, the FDA have approved the clinical implementation of FFR derived from computational modelling. Optimised methods of determining vFFR (e.g., 3D-QCA derived vFFR) have provided results in ˜4 min or near real time. Although early results have been promising, the precision of vFFR computation is limited by the accuracy by which the model represents the coronary and lesion geometry (imaging and reconstruction) and the physiological parameters (boundary condition tuning) on an individual patient basis. The final major barrier to a reliable vFFR tool is the application of a patient-specific tuning strategy to represent either hyperaemic flow or myocardial resistance.

Optimized treatment planning for stent implantation could be established using better imaging systems, especially in patients with comorbid conditions who were at a higher risk for cardiac events. A recent network meta-analysis clearly demonstrated the superiority of intravascular ultrasound (IVUS) and/or optical coherence tomography (OCT) versus coronary angiographic guidance (Buccheri et al. 2017). In particular, because angiography has known limitations in assessing vessel size and plaque burden, lesion calcium and eccentricity, stent expansion and geographic miss and complications, IVUS and OCT are now being used to answer questions that occur during routine PCI. OCT use is rapidly supplanting older technology, such as IVUS due to its 10× higher image resolution and faster image acquisition times, e.g., in Japan, OCT is used in ˜80% of all PCIs. Therefore, experts believe OCT analysis will be a critical tool, with automated image analysis urgently required.

One of the key benefits of using OCT over any other image modality is the ability to measure thin-cap fibroatheroma (TCFA), which is the rim of fibrous tissue separating the necrotic core of the plaque from the lumen of the artery. Rupture of the TFCA means the contents of the necrotic core spill into the bloodstream and cause blockages downstream. The most dangerous TCFA thickness is <65 μm and this can only be measured using OCT. TCFA thickness is one measure of risk, but the unpredictability of coronary artery disease is due to the behaviour of unstable vs stable plaques, with no methods currently available to assess plaque stability.

In addition to TCFA, many other biomarkers of plaque stability can be visualised and quantified on OCT. Current features of clinical interest are:
(1) lipid region;
(2) superficial calcium;
(3) deep calcium;
(4) plaque free wall;
(5) thrombus;
(6) macrophages;
(7) microchannels;
(8) cholesterol crystals; and of course
(9) thin cap fibro-atheroma.

Each of these contribute to the risk of plaque rupture causing heart attack and potential death. Despite the perceived importance of these features, currently they must be manually annotated and quantified on each OCT image (of which there is typically ˜500 per artery segment). This is not only hugely time consuming, but also introduces user variability.

Further to image-based biomarkers of plaque stability, there is growing evidence to suggest that biomechanical aspects are critical to plaque assessment. There are typically two forces considered from a biomechanical viewpoint; shear stress and structural stress.

Shear stress is the frictional force exerted on the inside of the vessel (i.e., of either an artery or vein) wall and on the plaque by the flowing blood. Computational fluid dynamics (CFD) is primary method used to calculate the shear stress acting on the inner walls of a patient's arteries or veins. A certain level of shear stress is required for normal physiological function and low shear stress is a well-established predictor of plaque progression and future clinical events. There are currently no commercial tools available to provide clinicians with usable vessel shear stress information.

The structural stress exerted on the vessel and plaque due to the blood pressure is also of major clinical value. A plaque will rupture when the structural stress exceeds the structural strength of the tissue. This has been a focus of major research efforts for many years and significant advances have been made. However, as with shear stress, there are currently no commercial tools available to clinicians that can provide these important data.

There are currently no methods available to semi-automatically or automatically analyse OCT image data beyond the lumen or calculate the important biomechanical forces that destabilise plaques and cause life-threatening plaque rupture (heart attacks). Furthermore, clinicians still require an intravascular imaging and software pre-PCI treatment planning tool that can comprehensively select the best stent size, length, and placement to minimise any further damage to the artery.

The uptake of ¹⁸F-Sodium Fluoride (¹⁸F—NaF) detected by positron emission tomography (PET) is associated with high-risk plaque coronary features and future clinical events (Joshi et al., 2014, ¹⁸F-fluoride positron emission tomography for identification of ruptured and high-risk coronary atherosclerotic plaques: a prospective clinical trial. Lancet, 383, 705-13; Lee et al., 2017, Clinical Relevance of (18)F-Sodium Fluoride Positron-Emission Tomography in Noninvasive Identification of High-Risk Plaque in Patients with Coronary Artery Disease. Circ Cardiovasc Imaging, 10). Regions of ¹⁸F—NaF uptake indicate microcalcification activity in the arterial wall and these regions develop into macrocalcifications in the future. Recently, it has been shown that ¹⁸F—NaF PET provides powerful independent prediction of fatal or nonfatal myocardial infarction (Kwiecinski et al., 2020, Coronary ¹⁸F-Sodium Fluoride Uptake Predicts Outcomes in Patients With Coronary Artery Disease. J Am Coll Cardiol, 75, 3061-74). In Kwiecinski et al., they converted ¹⁸F—NaF uptake to a comparable measure called Coronary Microcalcification Activity (CMA) which represents the overall disease activity in the vessel based on the volume and intensity of the ¹⁸F—NaF PET-activity in the vessel. Therefore, detecting, visualising, and quantifying ¹⁸F—NaF uptake as an early indicator of future adverse event has major clinical relevance. However, this imaging technique is expensive, only available in specialised centres and requires significant technical expertise to analyse the images. Furthermore, the patient is exposed to additional radiation and some are intolerant to the imaging radiotracer.

The ¹⁸F—NaF tracer, along with other blood borne particles, is transported via the blood stream and binds to early and active vascular calcifications (Irkle et al., 2015, Identifying active vascular microcalcification by ¹⁸F-sodium fluoride positron emission tomography. Nature Communications, 6, 7495). It therefore is influenced by hemodynamics (transport to the plaque site) and the distribution of plaques in the coronary arteries. Coronary plaques that are propagating/active are considered to be preferential binding sites for NaF, as microcalcification activity is occurring; triggered by cell death and inflammation (Chen and Dilsizian, 2013, Targeted PET/CT Imaging of Vulnerable Atherosclerotic Plaques: Microcalcification with Sodium Fluoride and Inflammation with Fluorodeoxyglucose. Current Cardiology Reports, 15, 364).

The blood supply to a coronary vessel has been shown to depend on geometric measurements, such as vessel diameter/calibre, vessel lengths and myocardial muscle mass (Zamir et al., 1992, Relation between diameter and flow in major branches of the arch of the aorta. J Biomech, 25, 1303-10; Choy and Kassab, 2008, Scaling of Myocardial Mass to Flow and Morphometry of Coronary Arteries. Journal of Applied Physiology (Bethesda, Md.: 1985), 104, 1281-1286). The propagation of coronary plaques changes local vessel calibres and shape, through remodeling (Libby and Theroux, 2005, Pathophysiology of coronary artery disease. Circulation, 111, 3481-8). This shape change is often described by geometric measures, such as area and eccentricity (Hausmann et al., 1994 Lumen and plaque shape in atherosclerotic coronary arteries assessed by in vivo intracoronary ultrasound. Am J Cardiol, 74, 857-63), and has been related to elevated structural stresses within plaques (Costopoulos et al., 2017 Plaque Rupture in Coronary Atherosclerosis Is Associated with Increased Plaque Structural Stress. JACC Cardiovasc Imaging, 10, 1472-1483). Plaque features have also been associated with both high and low endothelial wall shear stress (WSS) (Koskinas et al., 2009 The role of low endothelial shear stress in the conversion of atherosclerotic lesions from stable to unstable plaque. Curr Opin Cardiol, 24, 580-90), which is the fluid friction force acting on the endothelium. The value of WSS at a particular location is dependent on many factors, including blood supply, blood properties (e.g., viscosity), vessel calibre and luminal shape (e.g., eccentricity, curvature (Myers et al., 2001 Factors Influencing Blood Flow Patterns in the Human Right Coronary Artery. Annals of Biomedical Engineering, 29, 109-120)). Low WSS has been found to stimulate an atherogenic phenotype and promote arterial inflammation, where the expression of adhesion proteins and chemokines co-operate to capture leukocytes from the blood stream to the vessel (Malek et al., 1999 Hemodynamic shear stress and its role in atherosclerosis. Jama, 282, 2035-42; Lawrence et al., 1987 Effect of flow on polymorphonuclear leukocyte/endothelial cell adhesion. Blood, 70, 1284-90; and Gijsen et al., 2019, Expert recommendations on the assessment of wall shear stress in human coronary arteries: existing methodologies, technical considerations, and clinical applications. European Heart Journal, 40, 3421-3433). Furthermore, low WSS is present in regions of recirculating flow and low near-wall velocity, which helps to aggregate blood borne particles close to the endothelium (Gijsen et al., 2019, Expert recommendations on the assessment of wall shear stress in human coronary arteries: existing methodologies, technical considerations, and clinical applications. European Heart Journal, 40, 3421-3433). These hemodynamic mechanisms, and the associated endothelial dysfunction, help explain why plaque progression occurs in regions of low shear stress (Chatzizisis et al., 2008 Prediction of the Localization of High-Risk Coronary Atherosclerotic Plaques on the Basis of Low Endothelial Shear Stress. Circulation, 117, 993-1002; Stone et al., 2012, Prediction of Progression of Coronary Artery Disease and Clinical Outcomes Using Vascular Profiling of Endothelial Shear Stress and Arterial Plaque Characteristics: The PREDICTION Study. Circulation; Kumar et al., 2018, Low Coronary Wall Shear Stress Is Associated with Severe Endothelial Dysfunction in Patients with Nonobstructive Coronary Artery Disease. JACC Cardiovasc Interv, 11, 2072-2080; Yamamoto et al., 2017, Low Endothelial Shear Stress Predicts Evolution to High-Risk Coronary Plaque Phenotype in the Future. Circulation: Cardiovascular Interventions, 10, e005455; and Bourantas et al., 2019 Implications of the local hemodynamic forces on the phenotype of coronary plaques. Heart, heartjnl-2018-314086).

We consider the same environment to facilitate the detection of microcalcification activity through local NaF tracer residence, infiltration, and binding.

Attempts to use both shear stress and structural stress clinically are currently limited by significant difficulties such as automated image analyses, computational times, and lack of important boundary condition data for the simulations. No solutions to these problems have so far been described. Furthermore, quantitative data on ¹⁸F-NaF uptake (i.e., active microcalcification) is showing clinical promise, yet there exists a strong need to overcome the current requirements for expensive and inaccessible ¹⁸F-NaF PET hardware, the expertise in nuclear medicine and image analysis to access and interpret the ¹⁸F-NaF data, the issue of excessive radiation and patient intolerance to the radiotracer.

It is against this background that the present invention has been developed. In particular, the present invention seeks to overcome, or at least ameliorate, one or more of the deficiencies of the prior art mentioned above, or to provide the consumer with a useful or commercial choice.

### SUMMARY OF THE INVENTION

It is an object of the present invention to overcome or ameliorate at least one or more of the disadvantages of the prior art, or to provide a useful alternative.

One embodiment provides a computer program product for performing a method as described herein.

One embodiment provides a non-transitive carrier medium for carrying computer executable code that, when executed on a processor, causes the processor to perform a method as described herein.

One embodiment provides a system configured for performing a method as described herein.

The present invention is directed to a principal of general application in that it provides a method of measuring microcalcification activity in an artery (preferably a coronary artery) using anatomical measurements, image measurements, plaque measurements and blood flow/hemodynamic measurements. In particular the invention accommodates measurements of regions and lesions of interest in the vasculature that are healthy and/or diseased, and provides a means to identify/quantify/differentiate between these states. The list of possible inputs and outputs for use in the method are extensive with microcalcification prediction being the outcome that may be derived/possibly-intended from the method.

According to the invention, the inventors provide a method of measuring microcalcification activity in a blood vessel (preferably a coronary artery).

According to a first aspect of the invention, there is provided a method of predicting microcalcification activity in a vascular vessel. The vessel may comprise either an artery or a vein. The method may comprise the step of (a) measuring one or more of: the existence of and/or quantity of coronary plaques or visible markers of disease in a vascular tissue sample; and/or the existence of and/or quantity of healthy tissue in the vascular tissue sample; and/or one or more features that define an abnormal hemodynamic environment in a vessel; and/or one or more geometric features that are associated with vascular remodeling and which influence hemodynamics in a vessel, and/or one or more material properties that influence vascular hemodynamics. The method may further comprise the step of (b) calculating the microcalcification activity in the vessel as a function of the measurements taken in step (a).

According to a particular arrangement of the first aspect, there is provided a method of predicting microcalcification activity in a vascular vessel comprising either an artery or a vein, comprising the steps of:
(a) measuring one or more of:
   (i) the existence of and/or quantity of coronary plaques or visible markers of disease in a vascular tissue sample; and/or
   (ii) the existence of and/or quantity of healthy tissue in the vascular tissue sample; and/or
   (iii) one or more features that define an abnormal hemodynamic environment in a vessel; and/or
   (iv) one or more geometric features that are associated with vascular remodeling and which influence hemodynamics in a vessel, and/or
   (v) one or more material properties that influence vascular hemodynamics; and
(b) calculating the microcalcification activity in the vessel as a function of the measurements taken in step (a).

According to a second aspect of the invention, there is provided a method of predicting microcalcification activity in a vessel. The method may comprise the step of obtaining training data. The training data may consist of: general patient data comprising data relating to multiple patients and multiple data; and microcalcification activity data (μCA) from a plurality of patients at one or more anatomical locations. The method may further comprise the step of fitting a multivariate function/model by computing a function from inputted patient data and microcalcification activity data to estimate or predict μCA for new data [a_{new}, b_{new}, c_{new}, d_{new} . . . ] obtained in the same manner as the inputted training data set [A_{Tr}, B_{Tr}, C_{Tr}, D_{Tr}, . . . ], said function being:

$$μ {CA}_{prediction} = f ( {a}_{new} , {b}_{new} , {c}_{new} , {d}_{new} ⋯ )$$

The method may further comprise the step of evaluating the multivariate model by evaluating the previously fitted function to obtain a set of estimated values of microcalcification activity (μCA_{Est}) for a set of new function inputs. The method may further comprise the step of computing the error estimate using an error function Ef, by comparing the set of estimated values of microcalcification activity (μCA_{Est}) to the set of known/corresponding values of microcalcification activity data (μCA_{Te}) derived from the corresponding set of data that was collected for the same patients and used to generate function inputs, where:

$$Error = Ef ( μ {CA}_{Est} , μ {CA}_{Te} )$$

The method may further comprise the step of checking the error estimate to assess the suitability of the model.

According to a particular arrangement of the second aspect, there is provided a method of predicting microcalcification activity in a vessel, comprising the steps of:
obtaining training data consisting of:
(i) general patient data comprising data relating to multiple patients and multiple data; and
(ii) microcalcification activity data (pCA) from a plurality of patients at one or more anatomical locations;
fitting a multivariate function/model by computing a function from inputted patient data and microcalcification activity data to estimate or predict μCA for new data [a_{new}, b_{new}, c_{new}, d_{new} . . . ] obtained in the same manner as the inputted training data set [A_{Tr}, B_{Tr}, C_{Tr}, D_{Tr}, . . . ], said function being:

$$μ {CA}_{prediction} = f ( {a}_{new} , {b}_{new} , {c}_{new} , {d}_{new} … ) ;$$

evaluating the multivariate model by evaluating the previously fitted function to obtain a set of estimated values of microcalcification activity (μCA_{Est}) for a set of new function inputs;
computing the error estimate using an error function Ef, by comparing the set of estimated values of microcalcification activity (μCA_{Est}) to the set of known/corresponding values of microcalcification activity data (μCA_{Te}) derived from the corresponding set of data that was collected for the same patients and used to generate function inputs, where:

$$Error = Ef ( μ {CA}_{Est} , μ {CA}_{Te} ) ;$$

checking the error estimate to assess the suitability of the model.

According to a third aspect of the invention, there is provided a method for predicting microcalcification activity in a vessel. The method may comprise the step of receiving training data associated with one or more of: the existence of and/or quantity of coronary plaques or visible markers of disease in a vascular tissue sample; and/or the existence of and/or quantity of healthy tissue in the vascular tissue sample; and/or one or more features that define an abnormal hemodynamic environment in a vessel; and/or one or more geometric features that are associated with vascular remodeling and which influence hemodynamics in a vessel, and/or one or more material properties that influence vascular hemodynamics. The method may further comprise the step of determining one or more training features based on the training data values. The method may further comprise the step of determining one or more training labels associated with the one or more training features. The method may further comprise the step of building a predictive model, using a computer, for determining microcalcification activity in a vessel. Building the predictive model may include the step of inputting the one or more training features and the one or more training labels associated with the one or more training features to a machine learning algorithm. Building the predictive model may further include the step of determining a predictive model from the machine learning algorithm, the predictive model for receiving new data associated with a vessel; and determining a predictive label based on the new data.

According to a particular arrangement of the third aspect, there is provided a method for predicting microcalcification activity in a vessel comprising:
receiving training data associated with one or more of:
(i) the existence of and/or quantity of coronary plaques or visible markers of disease in a vascular tissue sample; and/or
(ii) the existence of and/or quantity of healthy tissue in the vascular tissue sample; and/or
(iii) one or more features that define an abnormal hemodynamic environment in a vessel; and/or
(iv) one or more geometric features that are associated with vascular remodeling and which influence hemodynamics in a vessel, and/or
(v) one or more material properties that influence vascular hemodynamics; determining one or more training features based on the training data values; determining one or more training labels associated with the one or more training features; building a predictive model, using a computer, for determining microcalcification activity in a vessel wherein building the predictive model includes:
   inputting the one or more training features and the one or more training labels associated with the one or more training features to a machine learning algorithm; and
   determining a predictive model from the machine learning algorithm, the predictive model for receiving new data associated with a vessel and determining a predictive label based on the new data.

According to a fourth aspect of the invention, there is provided a computer implemented method of measuring microcalcification activity in a vessel. The computer implemented method may comprise the step of (a) measuring one or more of: the existence of and/or quantity of coronary plaques or visible markers of disease in a vascular tissue sample; and/or the existence of and/or quantity of healthy tissue in the vascular tissue sample; and/or one or more features that define an abnormal hemodynamic environment in a vessel; and/or one or more geometric features that are associated with vascular remodeling and which influence hemodynamics in a vessel, and/or one or more material properties that influence vascular hemodynamics. The computer implemented method may further comprise the step of (b) using a trained machine learning model, calculating the microcalcification activity in the vessel as a function of the measurements taken in step (a).

According to a particular arrangement of the fourth aspect, there is provided a computer implemented method of measuring microcalcification activity in a vessel, comprising the steps of:
(a) measuring one or more of:
   (i) the existence of and/or quantity of coronary plaques or visible markers of disease in a vascular tissue sample; and/or
   (ii) the existence of and/or quantity of healthy tissue in the vascular tissue sample; and/or
   (iii) one or more features that define an abnormal hemodynamic environment in a vessel; and/or
   (iv) one or more geometric features that are associated with vascular remodeling and which influence hemodynamics in a vessel, and/or
   (v) one or more material properties that influence vascular hemodynamics; and
(b) using a trained machine learning model, calculating the microcalcification activity in the vessel as a function of the measurements taken in step (a).

The first machine learning model may comprise a first trained regression model.

The vessel may be one or more of a coronary artery, carotid artery, cerebral artery, aorta, peripheral artery, or vein.

According to a fifth aspect of the invention, a method of providing information for predicting the uptake of 18F-NAF in vascular tissues of a patient. The method may comprise the step of, using image processing means on patient image data, measuring vascular biomarkers indicative of the existence of and/or quantity of coronary plaques or visible markers of disease in the vascular tissue associated with cardiovascular disease progression. The method may comprise the further step of, using a processor, calculating the microcalcification activity in the vascular tissue as a function of the measurements.

According to a particular arrangement of the fifth aspect there is provided a method of providing information for predicting the uptake of 18F-NAF in vascular tissues of a patient, comprising: using image processing means on patient image data, measuring vascular biomarkers indicative of the existence of and/or quantity of coronary plaques or visible markers of disease in the vascular tissue associated with cardiovascular disease progression; and, using a processor, calculating the microcalcification activity in the vascular tissue as a function of the measurements.

According to a sixth aspect of the invention, there is provided a computer system comprising at least one processor; and at least one memory device storing patient data. The stored patient date may relate to: the existence of and/or quantity of coronary plaques or visible markers of disease in a vascular tissue sample; and/or the existence of and/or quantity of healthy tissue in the vascular tissue sample; and/or one or more features that define an abnormal hemodynamic environment in a vessel; and/or one or more geometric features that are associated with vascular remodeling and which influence hemodynamics in a vessel, and/or one or more material properties that influence vascular hemodynamics. The at least one processor may be configured for, using a trained machine learning model, calculating the microcalcification activity in the vessel as a function of the patient data.

According to a particular arrangement of the sixth aspect, there is provided a computer system comprising:
at least one processor;
at least one memory device storing patient data relating to:
(i) the existence of and/or quantity of coronary plaques or visible markers of disease in a vascular tissue sample; and/or
(ii) the existence of and/or quantity of healthy tissue in the vascular tissue sample; and/or
(iii) one or more features that define an abnormal hemodynamic environment in a vessel; and/or
(iv) one or more geometric features that are associated with vascular remodeling and which influence hemodynamics in a vessel, and/or
(v) one or more material properties that influence vascular hemodynamics; and wherein the at least one processor is configured for, using a trained machine learning model, calculating the microcalcification activity in the vessel as a function of the patient data.

The methods of any one of the above aspects may comprise measuring microcalcification activity in a coronary artery, carotid artery, cerebral artery, aorta, peripheral artery, or any vessel of interest, including veins.

According to a further aspect of the present invention, there is provided a method of predicting microcalcification activity in a vessel, comprising the steps of:
obtaining training data (Tr) consisting of:
   general patient data, [A_{Tr}, B_{Tr}, C_{Tr}, D_{Tr} . . . ], comprising data relating to multiple patients and multiple data which may further include image data and/or biomechanical data at one or more anatomic locations, and
   microcalcification activity data (μCA) from a plurality of patients at one or more anatomical locations;
fitting a multivariate function/model by computing a function from inputted patient training data and microcalcification activity data that can estimate/predict μCA for new data a_{new}, b_{new}, c_{new}, d_{new} . . . obtained in the same manner as the inputted training data set A_{Tr}, B_{Tr}, C_{Tr}, D_{Tr}, . . . , said function being:

$$μ {CA}_{prediction} = f ( {a}_{new} , {b}_{new} , {c}_{new} , {d}_{new} … ) ;$$

evaluating the multivariate model by evaluating the previously fitted function ƒ to obtain a set of estimated values of microcalcification activity (μCA_{Est}) for a set of new function inputs, which is a set of new General Patient test data (Te) that was not included in the training set, [A_{Te}, B_{Te}, C_{Te}, D_{Te} . . . ] where:

$$μ {CA}_{Est} = f ( {A}_{Te} , {B}_{Te} , {C}_{Te} , {D}_{Te} … ) ;$$

computing the error estimate using an error function Ef, by comparing the set of estimated values (Est) of microcalcification activity (μCA_{Est}) to the set of known/corresponding values of microcalcification activity data (μCA_{Te}) derived from the corresponding set of data that was collected for the same patients and used to generate function inputs [A_{Te}, B_{Te}, C_{Te}, D_{Te} . . . ], where:

$$Error = Ef ( μ {CA}_{Est} , μ {CA}_{Te} ) ;$$

checking the error estimate/assess the suitability of the model. If the error estimates meet a set of desired criteria, such as a required accuracy, precision, and sensitivity to input data where the error is low and the results are statistically significant, then the model may be considered fit for purpose, and used to predict microcalcification activity.

According to a further aspect of the invention, there is provided a non-transitory computer readable medium storing computer program instructions for measuring microcalcification activity in a vessel (preferably a coronary artery), the computer program instructions when executed by a processor cause the processor to perform operations, comprising:
(a) measuring one or more of:
   (i) the existence of and or quantity of a plaque or visible markers of disease in a vascular tissue sample;
   (ii) the existence of and or quantity of healthy tissue in the vascular tissue sample;
   (iii) one or more of the features that define an abnormal hemodynamic environment in a vessel;
   (iv) one or more of the geometric features that are associated with vascular remodeling and which influence hemodynamics in a vessel, and or
   (v) one or more of the material properties that influence vascular hemodynamics; and
(b) calculating the microcalcification activity in the vessel as a function of the measurements taken in step (a).

According to a further aspect of the invention, there is provided a non-transitory computer readable medium storing computer program instructions for measuring microcalcification activity in a vessel (preferably a coronary artery), the computer program instructions when executed by a processor cause the processor to perform operations, comprising:
obtaining training data consisting of:
   general patient data such as [A_{Tr}, B_{Tr}, C_{Tr}, D_{Tr}, . . . ], and
   microcalcification activity data (μCA), from a plurality of patients at one or more anatomical locations;
   fitting a multivariate function/model by computing a function from inputted patient data and microcalcification activity data that can estimate/predict μCA for new data obtained in the same manner as the inputted training data, said function being:

$$μ {CA}_{prediction} = f ( {a}_{new} , {b}_{new} , {c}_{new} , {d}_{new} … ) ;$$

evaluating the multivariate model by evaluating the previously fitted function ƒ to obtain a set of estimated values of microcalcification activity (μCA_{Est}) for a set of new function inputs, which is a set of new General Patient test data that was not included in the training set, [A_{Te}, B_{Te}, C_{Te}, D_{Te} . . . ] comprising data relating to multiple patients and multiple data which may further include image data and/or biomechanical data at one or more anatomic locations;

$$μ {CA}_{Est} = f ( {A}_{Te} , {B}_{Te} , {C}_{Te} , {D}_{Te} … ) ;$$

computing the error estimate, by comparing the set of estimated values of microcalcification activity (μCA_{Est}) to the set of known/corresponding values of (μCA_{Te}) using an error function Ef:

$$Error = Ef ( μ {CA}_{Est} , μ {CA}_{Te} ) ;$$

checking the error estimate/assess the suitability of the model. If the error estimates meet a set of desired criteria, such as a required accuracy, precision, and sensitivity to input data, then model may be considered fit for purpose, and used to predict microcalcification activity.

According to a further aspect of the invention, there is provided a non-transitory computer readable medium storing computer program instructions for measuring microcalcification activity in an artery in a patient, the computer program instructions when executed by a processor cause the processor to perform operations, comprising:
receiving a multivariate function/model comprising a function computed from a trained model of inputted patient data and microcalcification activity CA data that can estimate/predict μCA;
receiving general patient data of the patient consistent with the inputted patient data of the trained model; and computing values of microcalcification activity (μCA_{Est}) in the artery of the patient.

The methods of any one of the above aspects may further include one or more of the following features in any combination.

The method may comprise measuring microcalcification activity in a coronary artery, carotid artery, cerebral artery, aorta, peripheral artery, or any vessel of interest, including veins.

The patient data and/or the training data may comprise biomarker data relating to one or more features of clinical interest including, but not limited to: lipid region; superficial calcium; deep calcium; plaque free wall; thrombus; macrophages; microchannels; cholesterol crystals; or thin cap fibro-atheroma in relation to one or more blood vessels of the patient.

The patient data and/or the training data may comprise one or more of image data, including, but not limited to, OCT, angiography; computed tomography (CT); CT angiography image data. The image data may be referenced to a common reference frame or common co-ordinate system. The image data may be transformed into the common reference frame. The image data may provide a complete representation of the patient's imaged arterial tree. The representation may be a 2-dimensional and/or three-dimensional image representation.

The methods may comprise interpolation of the image data between image frames and/or between anatomical landmarks.

The methods may comprise performing a structural simulation at any position of interest along an imaged vessel. The image data may be segmented by a user to identify different regions of plaque and vessel walls in the imaged vessels.

The methods may comprise estimating the in vivo material properties based on ratios of tissue stiffness. The tissue stiffness ratios may be based on in vivo material properties similar to other areas of the patient's cardiovascular system.

The methods may comprise providing measures of vessel status including, but not limited to: endoluminal sheer stress; plaque structural stress; plaque feature analysis; microcalcification activity; virtual stenting; vessel wall feature analysis; thin cap measurement; multimodal imaging; vessel branches; fractional flow reserve; rapid timeframes; and VR virtualisation.

Microcalcification activity in a vascular vessel, for example an artery, such as a coronary artery, or a vein, may be measured using positron emission tomography (PET). In the methods, an arbitrary set of measurements may be fit to a model (via regression or machine learning techniques) to obtain a strict microcalcification activity outcome.

The existence and or quantity of vascular plaques may be measured based on measuring well-established geometric markers of disease from intravascular optical coherence tomography (OCT) images, specifically, the presence of lipid, calcium, and macrophages (bright spots) in the plaque. For example, measurements may be taken of the average Lipid Arc [° ], average Calcium Arc [° ], average bright spots. Additional geometric measurements indicative of disease relate to vessel diameter, area, volume, arterial wall/layer thicknesses, tortuosity and eccentricity, and all combinations of these measures. Similarly, these measurements may be obtained by any other image modality typical of clinical practice.

The existence and or quantity of healthy tissue may be measured based on measuring the amount of healthy arterial wall visible using intravascular OCT images, for example, plaque free wall (PFW) is inversely related to disease. For example, measurements may be taken of the average plaque-free wall arc [° ]. Similarly, this measurement may be obtained by any other image modality typical of clinical practice.

In the methods, measurements of abnormal hemodynamic environments, such as blood-borne particle residence or abnormal WSS may be estimated using computational fluid dynamic (CFD) simulations or other methods capable of estimating wall shear stress (WSS) directly via the imaging technique. For example by measuring low shear area (LSA): Area of Low WSS [%] or high shear area (HSA): Area of High WSS [%] or the average of WSS [Pa]. Additional hemodynamic-derived metrics may include, but not be limited to, oscillatory shear index (OSI), relative residence time (RRT), low and oscillatory shear (LOS), endothelial activation potential (ECAP), velocity-derived field functions (e.g., vorticity), pressure drop, or any gradient of previously mentioned metrics (e.g., gradient of WSS).

Measurements of geometric features associated with vascular remodeling and influence hemodynamics may be obtained from intravascular OCT images (circumference and eccentricity) and coronary computed tomography angiography (CCTA). For example, by measuring average circumference [mm](using OCT), average eccentricity (using OCT), arterial wall/layer thicknesses, and or ventricular muscle mass [g](using CT).

Material properties that influence hemodynamics such as % haematocrit may be measured during routine blood sampling and may be used to tailor the viscosity model used to calculate WSS in CFD.

The methods may enable the assessment of treatment options based on one or more metrics of interest.

In the methods, imaging modalities used in obtaining the measurements of the methods may include one or more of: computerized tomography (CT); magnetic resonance imaging (MRI); ultrasound; intravenous ultrasound (IVUS); optical coherence tomography (OCT); single-photon emission computerized tomography (SPECT), PET; or NaF PET.

The methods may comprise fitting processes for multivariate functions, for example parametric or non-parametric regression. A portion of the training data may be reserved as validation data during the fitting process. The validation data may be used to estimate prediction error for model selection. Non-parametric regression may comprise, for example, methods such as kernel regression and machine-learning support-vector machines. Parametric fitting may comprise using a parametric machine learning algorithm or a traditional optimisation method that finds the minima of an objective function (for example, “sum of square error”). For a non-linear function, a particular example of this suitable for use in the above methods may be a direct search method for multi-dimensional unconstrained minimisation such as the Nelder-Mead simplex method. Parametric optimization may leverage commonly used function forms relating to biological relationships, for example, allometric scaling functions.

Additional objectives, advantages and novel features will be set forth in the description which follow or will become apparent to those skilled in the art upon examination of the drawings and the ensuing detailed description of several non-limiting embodiments which follows.

### BRIEF DESCRIPTION OF THE FIGURES

Notwithstanding any other forms which may fall within the scope of the present invention, a preferred embodiment/preferred embodiments of the invention will now be described, by way of example only, with reference to the accompanying drawings in which:

FIG. 1 illustrates an overview of a machine learning system and method for predicting plaque stability to provide a Clinical Decision Support software application;

FIG. 2 illustrates the training and testing processes of the method of the invention;

FIG. 3 illustrates a graphical depiction of a measurement of vessel tortuosity;

FIG. 4 illustrates regions of endothelial shear stress in coronary segments below a specific threshold, in Pascals;

FIG. 5 illustrates a graphical depiction of an arc/angle measurement obtained from an OCT image;

FIG. 6 illustrates regions of endothelial shear stress in coronary segments below a specific threshold, in Pascals, and the corresponding ¹⁸F—NaF TBR in the segment;

FIG. 7 illustrates a graph of the correlation between microcalcification training data and model output data according to the methods disclosed herein;

FIG. 8 illustrates the correlation between microcalcification test data and model output data according to the methods disclosed herein;

FIG. 9 illustrates optimised fits for max. TBR using all measurements in Table 1;

FIG. 10 illustrates optimised fits for max. TBR using a subset of measurements (top); the average relative contributions of these five measurements to the model approximations (bottom);

FIG. 11 illustrates optimised fits for max. TBR using a subset of categorical measurements obtained from intravascular OCT images (top); the average relative contributions of the OCT measurements to the model approximations (bottom);

FIG. 12 illustrates an example of an overfit model of a neural network including a two-layer feed-forward network with sigmoid hidden neurons and linear output neurons; and

FIG. 13 illustrates a block diagram that illustrates an example computer system with which an embodiment of system 100 may be implemented.

### DETAILED DESCRIPTION OF THE INVENTION

It should be noted in the following description that like or the same reference numerals in different embodiments denote the same or similar features.

The present invention is based on the discovery that an arbitrary set of measurements can be fit to a model via regression or machine learning techniques to obtain a strict microcalcification activity outcome that is typically measured using NaF PET. The systems and methods disclosed herein describe the unexpected realization of correlating sodium fluoride (NaF) uptake with features linked to plaque anatomy, haemodynamic environment, etc, through use of AI modelling methods from patient imaging data utilising direct AI on the patient image data to identify and reconstruct the anatomy, which then is used to extract derivative data for use with a regression model to determine microcalcification activity in the patient's arteries.

Despite the immense clinical promise of determining microcalcification activity, obtaining the measure from the current method of sodium fluoride-PET is expensive, requires additional time to prepare for and acquire, results in complex images, and is not widely available. Therefore, it is unlikely if it will make it to routine clinical use. The systems and methods disclosed herein describe a framework for creating AI and regression models configured to provide the ability to determine information on microcalcification activity from data that does not involve sodium fluoride-PET, in a way that has not been previously considered or implemented. The systems and methods disclosed herein describe a solution to the technical difficulty of acquiring sodium fluoride-PET which addresses the long-term problem of identifying those most at risk of heart attack and those who will most benefit from intervention.

Thus, the present invention is directed to a principal of general application in that it provides a method of measuring microcalcification activity in a vessel (for example, a coronary artery) without the need to perform NaF PET imaging.

Features of the present invention are now more fully described in the following sections of this description, which set out non-limiting aspects, embodiments, and examples of the invention. This description is included for the purposes of exemplifying the present invention. The following description should not be understood as a restriction on the broad summary or disclosure of the invention as set out above.

### General

Those skilled in the art will appreciate that the invention described herein is susceptible to variations and modifications other than those specifically described. The invention includes all such variation and modifications. The invention also includes all of the steps, features, formulations, and compounds referred to or indicated in the specification, individually or collectively and any and all combinations or any two or more of the steps or features.

Each document, reference, patent application or patent cited in this text is expressly incorporated herein in their entirety by reference, which means that it should be read and considered by the reader as part of this text. That the document, reference, patent application or patent cited in this text is not repeated in this text is merely for reasons of conciseness.

Any manufacturer's instructions, descriptions, product specifications, and product sheets for any products mentioned herein or in any document incorporated by reference herein, are hereby incorporated herein by reference, and may be employed in the practice of the invention.

The present invention is not to be limited in scope by any of the specific embodiments described herein. These embodiments are intended for the purpose of exemplification only. Functionally equivalent products, formulations and methods are clearly within the scope of the invention as described herein.

The invention described herein may include one or more range of values (e.g., dosage, concentration etc.). A range of values will be understood to include all values within the range, including the values defining the range, and values adjacent to the range which lead to the same or substantially the same outcome as the values immediately adjacent to that value which defines the boundary to the range. Accordingly, unless indicated to the contrary, the numerical parameters set forth in the specification and claims are approximations that may vary depending upon the desired properties sought to be obtained by the present invention.

It is to be understood that both the foregoing general description and the following detailed description are exemplary and explanatory only and are not restrictive of the invention as claimed.

In this application, the use of the singular includes the plural unless specifically stated otherwise.

The articles “a” and “an” are used herein to refer to one or to more than one (i.e., to at least one) of the grammatical object of the article. By way of example, “an element” refers to one element or more than one element.

In this application, the use of “or” means “and/or” unless stated otherwise. Furthermore, the use of the term “including”, as well as other forms, such as “includes” and “included”, is not limiting. Also, terms such as “element” or “component” encompass both elements and components comprising one unit and elements and components that comprise more than one subunit unless specifically stated otherwise.

As used herein in the specification and in the claims, the phrase “at least one”, in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B”, or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

The term “about” is used herein to refer to quantities that vary by as much as 30%, preferably by as much as 20%, and more preferably by as much as 10% to a reference quantity, being indicative of and within the experimental error of the indicated value (e.g., within 95% confidence intervals for the mean) or within 10% of the indicated value (whichever is greater). The use of the word ‘about’ to qualify a number is merely an express indication that the number is not to be construed as a precise value. Mean all values of the variable and the indicated value of the variable, and when used to refer to a time interval representing a week, “about 3 weeks” is 17 to 25 days, and about 2 to 4 weeks 10 to 40 days.

Throughout this specification, unless the context requires otherwise, the word “comprise” or variations such as “comprises” or “comprising”, will be understood to imply the inclusion of a stated integer or group of integers but not the exclusion of any other integer or group of integers. It is also noted that in this disclosure and particularly in the claims and/or paragraphs, terms such as “comprises”, “comprised”, “comprising” and the like can have the meaning attributed to it in U.S. Patent law; e.g., they can mean “includes”, “included”, “including”, and the like; and that terms such as “consisting essentially of” and “consists essentially of” have the meaning ascribed to them in U.S. Patent law, e.g., they allow for elements not explicitly recited, but exclude elements that are found in the prior art or that affect a basic or novel characteristic of the invention.

For the purpose of this specification, where method steps are described in sequence, the sequence does not necessarily mean that the steps are to be carried out in chronological order in that sequence, unless there is no other logical manner of interpreting the sequence.

In addition, where features or aspects of the invention are described in terms of Markush groups, those skilled in the art will recognise that the invention is also thereby described in terms of any individual member or subgroup of members of the Markush group.

The terms “patient” and “subject” are used interchangeably and includes mammals and non-mammals, including primates, livestock, companion animals, laboratory test animals, captured wild animals, birds (including eggs), reptiles and fish. Thus, the term refers to, at least, monkeys, humans, pigs, cattle, sheep, goats, horses, mice, rats, guinea pigs, hamsters, rabbits, cats, dogs, chickens, turkeys, ducks, other poultry, frogs, and lizards.

The terms “treat” and “treatment” means the prevention of a disorder, disease, or disease to which such term applies, or the prevention or reduction of one or more symptoms of such disorder or disease. It includes therapeutic treatments, prophylactic treatments, and applications in which one reduces the risk that a subject will develop a disorder or other risk factor. Treatment does not require the complete curing of a disorder and encompasses embodiments in which one reduces symptoms, underlying risk factors or delays progression of the disorder.

Other definitions for selected terms used herein may be found within the detailed description of the invention and apply throughout. Unless otherwise defined, all other scientific and technical terms used herein have the same meaning as commonly understood to one of ordinary skill in the art to which the invention belongs.

The various methods or processes outlined herein may be coded as software that is executable on one or more processors that employ any one of a variety of operating systems or platforms. Additionally, such software may be written using any of a number of suitable programming languages and/or programming or scripting tools, and also may be compiled as executable machine language code or intermediate code that is executed on a framework or virtual machine.

In this respect, various inventive concepts may be embodied as a computer readable storage medium (or multiple computer readable storage media) (e.g., a computer memory, one or more floppy discs, compact discs, optical discs, magnetic tapes, flash memories, circuit configurations in Field Programmable Gate Arrays or other semiconductor devices, or other non-transitory medium or tangible computer storage medium) encoded with one or more programs that, when executed on one or more computers or other processors, perform methods that implement the various embodiments of the invention discussed above. The computer readable medium or media can be transportable, such that the program or programs stored thereon can be loaded onto one or more different computers or other processors to implement various aspects of the present invention as discussed above.

The terms “program” or “software” are used herein in a generic sense to refer to any type of computer code or set of computer-executable instructions that can be employed to program a computer or other processor to implement various aspects of embodiments as discussed above. Additionally, it should be appreciated that according to one aspect, one or more computer programs that when executed perform methods of the present invention need not reside on a single computer or processor, but may be distributed in a modular fashion amongst a number of different computers or processors to implement various aspects of the present invention.

Computer-executable instructions may be in many forms, such as program modules, executed by one or more computers or other devices. Generally, program modules include routines, programs, objects, components, data structures, etc. that perform particular tasks or implement particular abstract data types. Typically, the functionality of the program modules may be combined or distributed as desired in various embodiments.

Also, data structures may be stored in computer-readable media in any suitable form. For simplicity of illustration, data structures may be shown to have fields that are related through location in the data structure. Such relationships may likewise be achieved by assigning storage for the fields with locations in a computer-readable medium that convey relationship between the fields. However, any suitable mechanism may be used to establish a relationship between information in fields of a data structure, including through the use of pointers, tags or other mechanisms that establish relationship between data elements.

Also, various inventive concepts may be embodied as one or more methods, of which an example has been provided. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

### Embodiments of the Description

It should be noted in the following description that like or the same reference numerals in different embodiments denote the same or similar features.

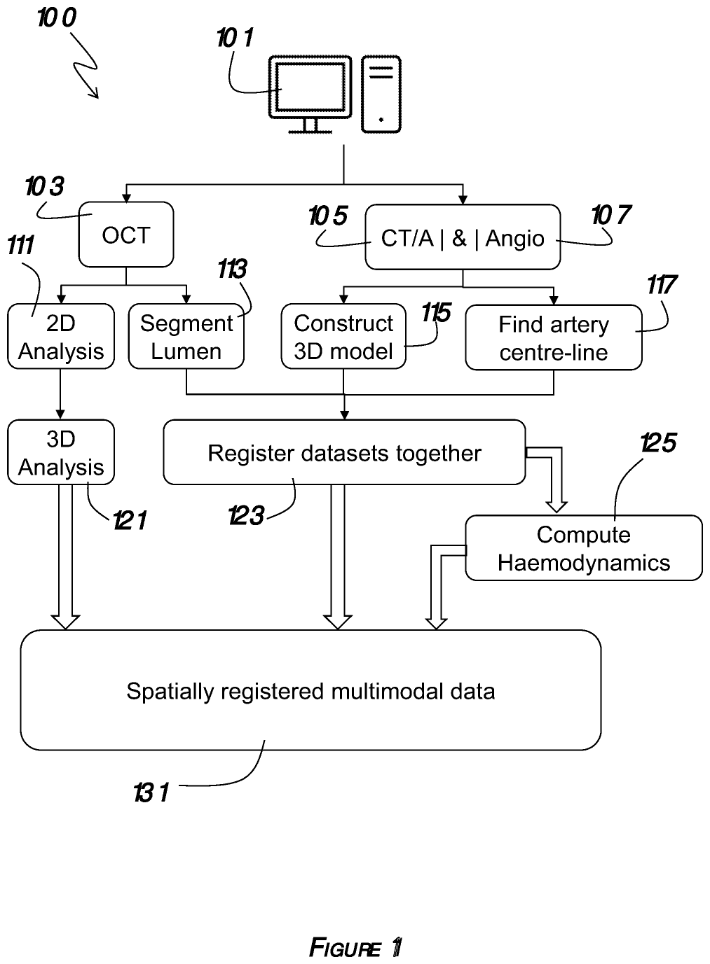

Disclosed herein are methods for predicting vascular plaque stability, particularly coronary artery plaque stability, and automatic computer-implemented systems and methods for predicting plaque stability to provide a Clinical Decision Support software application configured as a computer-implemented system 100 as shown in overview in FIG. 1 that uses a precision medicine patient-specific approach by analysing intravascular images and biomechanical computer models to provide comprehensive data on plaque stability. This creates unique patient-specific data, enabling clinicians to assess the risk of plaque rupture and tailor treatment plans, thus personalising healthcare. FIG. 1 illustrates the overall framework, described more clearly below.

The system 100 disclosed herein is particularly configured to enable rapid segmentation and annotation of intravascular patient image data (e.g., OCT), and links with computer models that calculate both shear stress and structural stress, to return otherwise unattainable data that has been shown to predict clinical events (Stone et al. 2016). Indeed, Professor Peter Stone from Harvard Medical School who has been championing the use of biomechanical data in predicting coronary events states that: “It has recently become clear that characterization of plaque risk based on anatomy alone is necessary but not sufficient to predict those high-risk plaques likely to destabilize and cause a new clinical event”. “If it were possible to calculate plaque stress in a routine and time-efficient manner in the catheterization laboratory, then this information could be enormously useful to identify the most high-risk plaques and inform management decisions”. The methods and computer-implemented systems disclosed herein provide a solution to this pressing clinical need.

The system 100 as disclosed herein, is a user-friendly, semi-automatic software tool for vessel and plaque feature assessment of OCT data in 2D and 3D capable of creating patient-specific 3D anatomical models from commercial OCT imaging systems. Importantly, system 100 can be used alongside any existing OCT imaging system.

Intravascular OCT 103 uses near-infrared light to create images of the inside of the coronary arteries. The technique delivers very high-resolution images (10-15 micron pixel size) and allows cardiologists to see the inside of an artery in 10 times more detail than if they were using intravascular ultrasound (IVUS; 100-150 micron pixel size), the next best technology, and up to 35 times better than state-of-the-art CT images (>350 micron pixel size). OCT also lets cardiologists clearly see the plaque inside an artery to measure build-up of fat and clot, and take precise measurements before and after placing stents.

OCT analyses: System 100 is configured to enable users to rapidly analyse plaque features and creates pixel-perfect segmentation (i.e., marking) tools for 2-dimensional analysis 111 of the lumen of the imaged artery and also segment the artery for example through a machine learning (ML) lumen contour segmentation routine to automate the user workflow. Edge detection algorithms based on state-of-the-art machine learning tools (deep learning using capsules) automatically obtain artery contours and data on artery size, shape, and position data. Current tools are time-consuming and manual (e.g., slice by slice, >1000 slices/patient). Hence, system 100 can provide considerable time-saving in comparison to current tools of plaque assessment including automatic lumen contour segmentation. Typical performance of the system 100 observed indicate that average arterial segmentation is comparable to the current state-of-the-art machine learning models but significantly faster to process the images, thus 100 creates segmentations that are indistinguishable from that obtained by a human operator and are reproducible with real time or near-real-time processing.

Current features of clinical interest include: Lipid region; superficial calcium; deep calcium; plaque free wall; thrombus; macrophages; microchannels; cholesterol crystals; and thin cap fibro-atheroma. Each of these contribute to the risk of plaque rupture or erosion causing heart attack and potential death. Each of these biomarkers are available within system 100, with the position of every data point stored in 3D for use during 3D registration and mapping of information between different 3D workspaces. Additional features can be included when and where relevant.

Once the OCT image analysis is complete, the user can covert the 2D image data into 3D for additional analysis 121. This converts the OCT data into a 3D reconstruction.

Software mapping: In most clinical scenarios, the OCT image data will be acquired alongside another modality, such as CT 105 and/or angiography 107. System 100 is adapted to merge high resolution OCT 103 with lower resolution angiography (angio) 107 or computed tomography (CT) or CT angiography (CTA) 105. CT data 105 or angiography 107 is readily converted 115 to a 3-dimensional model of the arterial tree providing data relating to the centre-line of the arteries in the imaged arterial tree. The image data from the different imaging modalities may be transformed into a common reference frame or co-ordinate system such that the combination of OCT data 103, CT/A data 105, and angio data 107/provides a complete representation of the imaged arterial tree including the left ventricle muscle mass obtained from the CT/A data. Once individual data sets are obtained, system 100 is configured to register the data from different investigations together in the same x-y-z workspace, such that all the different modes of data are qualitatively and quantitatively relatable to each other. Once the OCT data 103 is merged with angio data 107 or CT data 105, biomechanical simulations are possible. The result is a 3D geometry with exquisite detail in the region of OCT and novel 3D quantitative pathology data, unobtainable with any existing commercial software.

If for some reason there is only one particular mode of data available, the user can often still perform many of the functions in system 100, but will experience some limitations in the available analysis methods, for example, if only OCT data 103 is available, the main limitation is that calculations of shear stress will not be reliable due to the lack of 3D centreline data from CT/A or angio data.

The software mapping process performed by system 100 is configured to automatically interpolate the position of OCT image frames between anatomical landmarks. These landmarks (e.g., a vessel branch point) may be acquired from other medical imaging methods (e.g., CT/A or angio data). Furthermore, the CT data-set may be leveraged to acquire the left ventricular muscle mass which is used to improve the boundary conditions of CFD simulations.

Biomechanical simulation—structural stress: At any position along the imaged vessel, a structural simulation can be performed. These simulations are performed directly on the 2D OCT image where the image is segmented by the analyst/user using semi-automatic tools available within system 100 to identify the different regions of the plaque and vessel wall. As it is impossible to accurately know the in vivo material properties, system 100 uses a strategy based on ratios of tissue stiffness, similar to other areas of the cardiovascular system. This is a clinically-translatable method and unique to system 100.

Microcalcification estimator: Presence of microcalcification in coronary artery vessels is a predictor of future clinical events. This microcalcification activity is imaged and quantified using the uptake of the radiotracer ¹⁸F-sodium fluoride (NaF) on PET/CT. However, NaF-PET/CT imaging is expensive, not widely available, requires significant technical expertise to analyse the images and also introduces more radiation to the patient. Disclosed here in is a novel formula that predicts the uptake of NaF into the vessel wall and plaque, and thus predicts the microcalcification activity. It has also been surprisingly found that this formula significantly correlates with the in vivo uptake of NaF.

The systems and methods disclosed herein enable customisation of healthcare by giving patients and their doctors a predictive assessment of the chance of a clinical event based on detailed OCT, biomechanical modelling and microcalcification activity of their arteries. This will enable better preventative methods to be tested in patients identified as high risk and help de-escalate therapies for those at low risk, shifting away from the current ‘one size fits all’ approach used in hospitals. The system enables fast qualitative and unrivalled quantitative offline analysis of OCT data, and delivers biomechanical and microcalcification activity data that cannot be obtained via other commercial means, thus providing a new suite of patient-specific tools to cardiologists.

### Advantages

The computer implemented system 100 as disclosed herein is configured to provide clear benefits and advantages of common OCT image analysis tools which are integrated with OCT scanning equipment, and even provides clear advantages over third party available OCT software analysis tools and is configured to provided relevant measures of vessel status, from anatomical to functional, including:

Endoluminal Shear Stress (ESS), that is, the frictional biomechanical force acting on the innermost lining of the vessel, is a known predictor of plaque development, progression, and clinical event. Several research tools exist for calculating ESS from angio and CT-based 3D reconstructions (with or without the addition of OCT or IVUS) however most are cumbersome, require expertise in computational fluid dynamics, and lengthy computational time (e.g., 1-2 days on a typical workstation). System 100 utilises a hybrid approach to computing ESS that returns data within clinically-usable timeframes.

Plaque Structural Stress (PSS) is the force per unit area acting on the plaque. Plaque rupture occurs when the PSS exceeds the plaque cap strength, with PSS also having an impact on cell activities linked to plaque remodeling, inflammation, erosion, cell multiplication and other activities related to plaque progression and stability. In vivo plaque rupture data shows that in over 80% of cases, the location of maximum PSS coincides with rupture site. Despite there being data on the importance of PSS, it remains a research tool and is not built into any commercial software aimed at plaque analysis. This is in part due to the lack of knowledge around patient-specific material properties. System 100 circumvents this by using the principal of static determinacy, something that has been widely exploited in other cardiovascular diseases, such as aneurysms (Joldes et al. 2017), but not yet in coronary artery disease.

Plaque feature analysis is where OCT really stands out. The superior resolution over other modalities means that it can identify and quantify features of the plaque wall down to the cellular level (e.g., presence of macrophages). System 100 has built in sophisticated tools for the fast extraction of these features.

Microcalcification activity in the plaque wall is emerging as a strong non-invasive indicator of future clinical event. This activity is measured by the uptake of the radiotracer ¹⁸F-sodium fluoride (NaF) on positron emission tomography (PET) images. However, PET imaging is expensive, is not readily accessible, is difficult to interpret and also introduces the patient to more radiation. Disclosed herein is a novel method for the prediction of the uptake of NaF, and thus potentially the likelihood of a future clinical event, within a segment of an artery and without the need for PET imaging.

Virtual stenting is possible in the platform of system 100. As OCT offers unrivalled image resolution, stent planning is inherently more accurate. By determining accurate vessel dimensions, accurate stent selection is possible. Then, by selecting the appropriate 3D stent geometry in system 100, the stent can be virtually placed into the vessel at the desired location, after which the flow simulation can be performed. This allows data on stent performance prior to surgery.

Vessel wall feature analysis is similar to plaque analysis and the unrivalled image resolution means that the vessel wall can be rapidly identified and quantified. system 100 has developed automatic tools to segment the lumen based on deep learning (artificial intelligence, AI) that currently outperforms the state-of-the-art.

Thin cap measurement. Again, the image resolution is the key factor. Thin caps become clinically dangerous when they are <65 microns in thickness. Due to the resolution, OCT is the only modality capable of measuring this biomarker of risk.

Multimodal imaging. System 100 is configured to handle whatever image data is available to the clinician. CT and angiogram images, even using vFFR, cannot provide accurate information on plague progression, erosion, and rupture. The ideal scenario involves a combination of imaging modalities (e.g., CCTA and OCT) however if the clinician wants a reduced analysis performed using a single image modality (e.g., CCTA or angio), that is possible with system 100.

Vessel branches are included in the analysis in system 100. This provides true information on flow within arterial segments and accounts for branching flow along the vessel.

Fractional Flow Reserve (FFR) is the ratio of pressures upstream and downstream of a stenosis. If the pressure difference is greater than a certain threshold (e.g., 30%), intervention will be considered. FFR using only image data is the current ‘hot topic’ in cardiology as it enables measurements of FFR without the need for any induction of hyperaemic flow (forced increase in flow) or the presence of a pressure-measuring wire; two of the major factors limiting the uptake of standard FFR. Furthermore, image-based FFR has other major benefits. FFRCT (i.e., HeartFlow) only requires CT and thus is entirely non-invasive, but takes a lot longer to calculate (several hours turnaround time through HeartFlow). Most patients will receive angiography (invasive imaging) during routine care and FFR based on angiography is much faster and cheaper (i.e., VIRTUheart and CAAS).

FFR based on a combination of OCT data and angiography data (or only OCT if angiography data is not available) is also possible.

Rapid timeframes. System 100 is designed to work within clinical timeframes and is aiming to be ‘push button’. The methods used in system 100 have been verified so that the data produced using our efficient simulation strategy (i.e., minutes of CPU time) produces data comparable to that of a much longer simulation (i.e., days of CPU time).

VR visualisation. Outputs from system 100 are also VR-compatible to provide an immersive view of the problem and the resulting data.

### Aspects of the Invention

In a first aspect of the present invention, there is provided a method of measuring microcalcification activity in an artery (preferably a coronary artery), comprising the steps of:
(a) measuring one or more of:
   (i) the existence of and or quantity of coronary plaques or visible markers of disease in a vascular tissue sample;
   (ii) the existence of and or quantity of healthy tissue in the vascular tissue sample;
   (iii) one or more of the features that define an abnormal hemodynamic environment in a vessel;
   (iv) one or more of the geometric features that are associated with vascular remodeling and which influence hemodynamics in a vessel, and or
   (v) one or more of the material properties that influence vascular hemodynamics; and
(b) calculating the microcalcification activity in the vessel as a function of the measurements taken in step (a).

In an embodiment of the invention, microcalcification activity in a vascular vessel, for example an artery, such as a coronary artery, or a vein, is measured using positron emission tomography (PET).

According to the invention an arbitrary set of measurements can be fit to a model (via regression or machine learning techniques) to obtain a strict microcalcification activity outcome that is typically measured using NaF PET.

In an embodiment of the invention, the existence and or quantity of vascular plaques is measured based on measuring well-established geometric markers of disease from intravascular optical coherence tomography (OCT) images, specifically, the presence of lipid, calcium, and macrophages (bright spots) in the plaque. For example, measurements are taken of the average Lipid Arc [° ], average Calcium Arc [° ], average bright spots. Additional geometric measurements that are indicative of disease relate to vessel diameter, area, volume, arterial wall/layer thicknesses, tortuosity and eccentricity, and all combinations of these measures. Similarly, these measurements can be obtained by any other image modality typical of clinical practice.

In another embodiment of the invention, the existence and or quantity of healthy tissue is measured based on measuring the amount of healthy arterial wall visible using intravascular OCT images; plaque free wall (PFW) is inversely related to disease. For example, measurements are taken of the average plaque-free wall arc [° ]. Similarly, this measurement can be obtained by any other image modality typical of clinical practice.

In a further embodiment of the invention, measurements of abnormal hemodynamic environments, such as blood-borne particle residence or abnormal WSS will be typically estimated using computational fluid dynamic (CFD) simulations or other methods capable of estimating wall shear stress (WSS) directly via the imaging technique, such as MRI. For example, by measuring low shear area (LSA): Area of Low WSS [%] or high shear area (HSA): Area of High WSS [%] or the average of WSS [Pa]. Additional hemodynamic-derived metrics include, but are not limited to, oscillatory shear index (OSI), relative residence time (RRT), low and oscillatory shear (LOS), endothelial activation potential (ECAP), velocity-derived field functions (e.g., vorticity), pressure drop, or any gradient of previously mentioned metrics (e.g., gradient of WSS).

In yet another embodiment of the invention, measurements of the geometric features that are associated with vascular remodeling and influence hemodynamics are preferably obtained from intravascular OCT images (circumference and eccentricity) and coronary computed tomography angiography (CCTA). For example by measuring average circumference [mm](using OCT), average eccentricity (using OCT), arterial wall/layer thicknesses, and or ventricular muscle mass [g](using CT).

In yet another embodiment of the invention, material properties that influence hemodynamics such as % haematocrit is measured during routine blood sampling and can be used to tailor the viscosity model used to calculate WSS in CFD. Other methods of measuring/estimating % haematocrit also apply here.

In a preferred embodiment of the invention the microcalcification activity as determined by step (b) of the method enables the assessment of treatment options based on one or more metrics of interest.

In a form of the invention, imaging modalities used in obtaining the measurements of the method include computerized tomography (CT), magnetic resonance imaging (MRI), ultrasound, intravenous ultrasound (IVUS), optical coherence tomography (OCT), single-photon emission computerized tomography (SPECT), PET and NaF PET. Preferably measurements are obtained using NaF PET.

For a given set of measurements, belonging to any of the categories (i) through to (v), obtained from single or multiple sources (e.g., coronary imaging modalities), the function to estimate microcalcification activity may be obtained. Presently, explicitly defined formula have been used to describe the contribution of an arbitrary number of categorical measurements to microcalcification activity (measured using ¹⁸F—NaF PET), fitted using a standard optimisation (error minimisation) method.

These formula are described in the following example functions for P (microcalcification activity (μCA) prediction), of forms P₁ to P₆ use the categorical measurements (A₁, . . . , Eₙ) as arguments and lower-case letters describe the coefficients determined using fittings methods (e.g., via iterative optimisation methods). An offset value (Constant) may also be determined during fitting. The subscript n is the highest coefficient or measurement index of the function arguments with within each category (A-E). Note, with respect to previous/other descriptions/presentations of function arguments in this document (i.e., general patient data) the function arguments here are sorted into categories. This is done to describe how different function forms might aggregate related measurements to minimise the number fitting coefficients in the parametric functions. The equations may be implemented/fit ignoring measurements and coefficients of a given category if no such data exists or is excluded. For brevity, the equations written here describe the intermediate/repeating arguments/terms/categories as ‘ . . . ’:

$${P}_{1} ( {A}_{1} , … , {E}_{n} ) = {{a}_{1} ( {A}_{1} )}^{{α}_{1}} + … + {{a}_{n} ( {A}_{n} )}^{{α}_{n}} + … + {{e}_{1} ( {E}_{1} )}^{{ε}_{1}} + … + {{e}_{n} ( {E}_{n} )}^{{ε}_{n}} + Constant \qquad ( 1 )$$

$${P}_{2} ( {A}_{1} , … , {E}_{n} ) = a ( {A}^{α 1} \times … \times {A}^{α n} ) + … + 1 n \\ e ( {E}^{ε 2} \times … \times {E}^{ε n} ) + Constant 1 n \qquad ( 2 )$$

$${P}_{3} ( {A}_{1} , … , {E}_{n} ) = {a ( {A}_{1} \times … \times {A}_{2} )}^{α} + … + {e ( {E}_{1} \times … \times {E}_{n} )}^{ε} + Constant \qquad ( 3 )$$

$${P}_{4} ( {A}_{1} , … , {E}_{n} ) = {k ( {A}_{1} \times … \times {A}_{n} )}^{α} \times … \times {( {E}_{1} \times … \times {E}_{n} )}^{ε} + Constant \qquad ( 4 )$$

$${P}_{5} ( {A}_{1} , … , {E}_{n} ) = {( {a}_{1} {A}_{1} + … + {a}_{n} {A}_{n} )}^{α} \times … \times {( {e}_{1} {E}_{1} + … + {e}_{n} {E}_{n} )}^{ε} + Constant \qquad ( 5 )$$

$${P}_{6} ( {A}_{1} , … , {E}_{n} ) = {( {a}_{1} {A}_{1} + … + {a}_{n} {A}_{n} + {b}_{1} {B}_{1} + … + {b}_{n} {B}_{n} )}^{ω} + {c ( {C}_{1} \times … \times {C}_{n} )}^{γ} + {k ( {D}_{1} \times … \times {D}_{n} )}^{δ} \times {( {E}_{1} \times … \times {E}_{n} )}^{ε} + Constant \qquad ( 6 )$$

Form P₁ assumes all measurements contribute to the function independently (unique coefficients).
Form P₂ sees measurements of a given category given the same proportionate scaling coefficient, and are multiplied with unique exponents.
Form P₃ simplifies form P₂ by using the same exponent for measurements of a given category.
Form P₄ and P₅ provide examples of forms that are categorically multiplicative, however, these forms may compound measurement errors and are sensitive to measurements with values of zero (or extreme values).
P₆ exemplifies forms where different groupings of categories may be used. Measurements from categories A and B may be closely related, and share a unique exponent, while simulations results (category C) are considered exclusive.

Other finite combination of mathematical expressions may be used or generated for further optimisation of the method as discussed below. However, all of these functions, at most, include a single exponent coefficient per measurement to simplify the fitting process. The equations (notably P₁) leverage the power law formula that has been used to describe allometric scaling relationships throughout biology—including coronary artery blood supply. Simple power law relationships appear in fluid dynamics, with quantities such as flow, pressure and friction/WSS being dependent on radius in analytical solutions to the Navier-Stokes equations for (the physiologically relevant) internal pipe flow.

Optimised (parametric) fitting: To determine the coefficients in scalar equation(s) such as for P (above), the sum of the square error may be minimised, following the Nelder-Mead simplex algorithm: a direct search method for multi-dimensional unconstrained minimization:

$$ε = \sum {( - \vec{P} )}^{2} \qquad ( 7 )$$

Where, [symbol] is the vector of measured NaF uptake data and P is the vector containing the estimated values. This estimated value is computed using the scalar equation for P, for each sample. For the first iteration of the algorithm the coefficients in P are guessed (or set to arbitrary random values). After each iteration, the coefficients are updated until a minima is found and the algorithm terminates. This occurs when the error changes less than a specified tolerance. To assist the algorithms' ability to find the optimal set of coefficients, the input data (arguments of P) are normalised by their mean value.

Before providing specific examples, the main aspects to the methodology/framework are explained more broadly below. Central to this is the training and testing of the prediction model. After this the model may be considered fit for use.

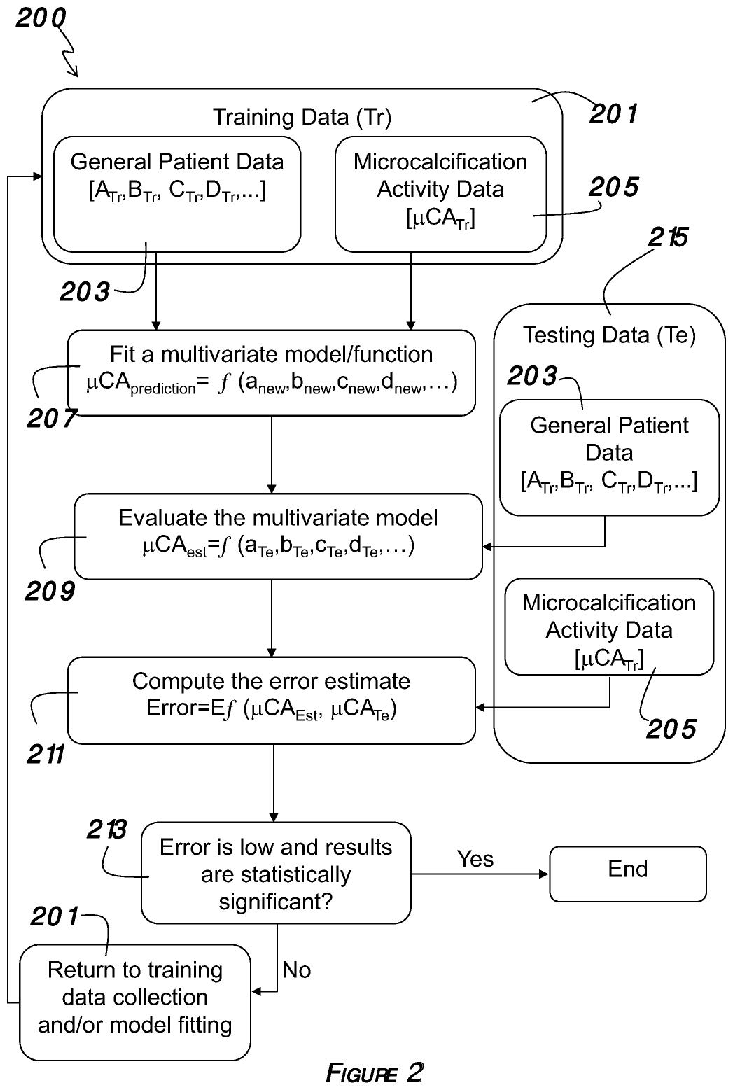

Broadly the process 200 involves the following steps as depicted in FIG. 2:

Step 1: Obtain 201 training data consisting of:
General patient data 203 such as [A_{Tr}, B_{Tr}, C_{Tr}, D_{Tr} . . . ] comprising data relating to multiple patients and multiple data which may further include image data and/or biomechanical data at one or more anatomic locations, and
Microcalcification activity data 205 (μCA), from multiple patients at one or more anatomical locations.

Step 2: Fitting 207 a multivariate function/model which involves computing a function from inputted patient data and microcalcification activity data that can estimate/predict μCA for new data obtained in the same manner as the inputted training data, said function being:

$$μ {CA}_{prediction} = f ( {a}_{new} , {b}_{new} , {c}_{new} , {d}_{new} … )$$

Step 3: Evaluate 209 the multivariate model by evaluating the previously fitted function ƒ to obtain a set of estimated values of microcalcification activity (μCA_{Est}) for a set of new function inputs, which is a set of new General Patient test data that was not included in the training set, [A_{Te}, B_{Te}, C_{Te}, D_{Te} . . . ].

$$μ {CA}_{Est} = f ( {A}_{Te} , {B}_{Te} , {C}_{Te} , {D}_{Te} … )$$

Step 4: Compute 211 the error estimate, by comparing the set of estimated values of microcalcification activity (μCA_{Est}) to the set of known/corresponding values of (μCA_{Te}) using an error function Ef:

$$Error = Ef ( μ {CA}_{Est} , μ {CA}_{Te} )$$

Step 5: Check the error estimate/assess the suitability of the model 213. If the error estimates meet a set of desired criteria, such as a required accuracy, precision, and sensitivity to input data, then model may be considered fit for purpose, and used to predict microcalcification activity.

Where the error is low and the results are statistically significant then an of estimate microcalcification activity will be obtained. Where the error is not low and or the results are not statistically significant the further training is required by the addition of further training data.

The following information presents background information on each aspect of the training and testing processes listed in FIG. 2.

Step 1(i)—General Patient Data 203 [a. B, C, D . . . ]

General patient data is derived from multiple patients and multiple data types and includes image data and or biomechanical data at one or more anatomical locations.

Preferably, general patient data includes patient data that is relevant to the estimation of microcalcification activity: including information that is likely to influence microcalcification activity as detected/measured using ¹⁸F-sodium fluoride (¹⁸F-NaF) positron emission tomography (PET).

There is no constraint on the number of data sets collected for fitting or the categorisation data in each data set. For example, all measurements could be geometric in nature, or none may be. However, they should reasonably correspond to metrics/measurements that are expected to be associated with atherosclerotic processes, and therefore with microcalcification activity, and also be attainable from clinical practice and/or through image processing of medical imaging modalities using common image processing means that are used, thus allowing the methodology to be readily applicable.

In a particular arrangement, a geometric measure corresponds to image-based diameter measurements (a standard measure of vessel patency/health) in a particular vessel that is prone to the calcification process, with extreme diameters being associated with unhealthy vasculature, and vessel size also being expected to influence the surface area available for ¹⁸F—NaF tracer transport to binding sites. The geometric measurements are obtained by image processing of patient image data including one or more of computer tomography, optical coherence tomography, intravascular ultrasound, x-ray angiography, magnetic resonance image or PET imaging.

Aside from direct anatomical measurements of geometry, measurements of vessel health or disease burden (e.g., coronary calcium score) are relevant, as well as patient-specific measurements that have been demonstrated to underpin cardiovascular disease progression. These measurements include, for example, both image-based (i.e., computer tomography, optical coherence tomography, intravascular ultrasound, x-ray angiography, magnetic resonance image or PET imaging measurement) and non-image-based measurements, such as patient history or blood sample data. Other measurements of relevance are biomechanical measurements, such as, for example, measurements of blood pressure, blood flow rate or localised hemodynamic characteristics, and tissue stresses. These types of metrics are expected to play a role in the ¹⁸F—NaF tracer transport to binding sites and have been broadly associated with cardiovascular disease progression.

The collected data may optionally undergo transformation/scaling before being used in for model fitting process to remove improve the performance of the fitting algorithms.

Step 1(ii)—Microcalcification Activity Data 205 (μCA)

¹⁸F—NaF PET image data obtained at one or more measurement scales from the PET scan of the patient's heart or other vascular region. This data should be collected for a plurality of patients and is the dependent variable in the fitting of the multivariate function/model (the step 207 in FIG. 2).

Following standard methods for measuring PET data, the recorded ¹⁸F—NaF PET data is recorded as an evaluation of the standardized uptake value at each sample region/location. This value is preferably adjusted (normalised) for blood pool activity, by measuring/evaluating the standardized uptake value at a reference location. An example of this would be to take the mean from regions of interest in the right atrium. In doing this, the PET measurement process is then standardised across patients, and provides a measurement of a tissue to background ratio (TBR). In the coronary arteries, ¹⁸F—NaF PET is often reported as either TBR or other similar measurements of uptake, see, for example, Coronary Microcalcification Activity (CMA) (Kwiecinski, J. et al. J Am Coll Cardiol. 2020; 75(24):3061-74).

Furthermore, the measurement scale refers to the method for which the ¹⁸F—NaF PET data is sampled from the medical images. This data could be taken as the maximum value within a region of the patient's vasculature. Alternatively, it could be sampled per discrete length interval/regions of interest along a patients' blood vessels. Examples of this could be sampling the data every 5 cm along the vessel centreline or sampling the data between bifurcations or sampling the data on every nth image, or sampling the maximum value of the data per vessel or predetermined anatomical segment/section. The data may also be mapped as a continuous function by measuring it with respect to a continuous variable, such as spatial dimension (e.g., axial distance in the medical image stack or distance along a coronary artery centreline). Obtaining the data this way allows the continuous function to be evaluated in a particular way (not-predetermined/during data collection) before the fitting step (e.g., taking the maximum or average value of the function in a particular region/interval). Furthermore, the locality of any sampled data points (e.g., the spatial dimension) may be considered as an independent variable in the fitting of a multivariate function. This allows for the fitted multivariate function to evaluate the spatial dependence of microcalcification activity measured using ¹⁸F—NaF PET. In this processes, the general patient data benefits from a similar spatial discretisation if also obtained from medical imaging.

To improve the spatial/anatomic localisation of the measurements the PET images are preferably co-registered with another image source (with a secondary/clearer representation of the patient-specific anatomy), such as contrast-enhanced computed tomography (to improve the appearance of the blood vessels) to improve the recording of spatial data associated with each ¹⁸F—NaF PET sample. Motion correction algorithms, e.g., elastic motion correction, may also be used to better present the PET-image data to aid with this process.

The collected data may optionally undergo transformation/scaling before being used in for model fitting process to remove improve the performance of the fitting algorithms.

### Step 2—Fitting 207 a Multivariate Function/Model

The fitting process 207 can be performed using any method for fitting a multivariate function as would be appreciated by the skilled addressee, such as, for example, parametric or non-parametric regression. Examples in the present specification include the Nelder-Mead simplex method, however alternate optimisation methods are available and would also be suitable as would be readily appreciated by the skilled addressee, where the same or similar coefficients are to be expected. In all cases, however, training and testing data is required irrespective of the whether or not a machine learning method is utilised for fitting process 207. Non-parametric regression is favoured as the form of the predictor equation (function fin FIG. 2) does not have a predetermined form, but is determined/constructed from information derived from the data being fit. However, because of this, it requires more data than parametric regression. It requires that some of the training data 201, as presented in FIG. 2, to be reserved as validation data 215 during the fitting process. While the majority of the training data 201 may be used to fit the model, this validation set (or subset) 215 is used to estimate prediction error for model selection. Categories of non-parametric regression include, for example, methods such as kernel regression and machine-learning support-vector machines. On the other hand, in parametric fitting the form of the function is assumed/predetermined and a method is used to learn/determine the coefficient(s) of the function. This could be done using a parametric machine learning algorithm or a traditional optimisation method that finds the minima of an objective function (for example, “sum of square error”). For a non-linear function, a particular example of this suitable for use in method 200 would be a direct search method for multi-dimensional unconstrained minimisation such as the Nelder-Mead simplex method. When performing parametric optimization (i.e., for equations 1-6), the form of the function being fit may benefit from leveraging commonly used function forms that have been used to describe relationships throughout biology—such as allometric scaling functions.

### Step 3—Evaluate 209 the Multivariate Model

The evaluation 209 of the model, as shown in FIG. 2, is key to testing the accuracy and suitability of the model. The model preferably is evaluated for a set of test data (general patient data: the model inputs/arguments; A_{Te}, B_{Te}, C_{Te}, D_{Te} . . . ) collected in the same way the training data was. This data should, preferably, be acquired from a broad set of patients from multiple sites and be sufficient in size to ensure that the fitted model does not suffer from simple errors, such as sensitivity during extrapolation (non-physical values obtained for data outside the range of the training data). The testing set 215 should not contain any of the general patient data 203 that was used during training 201. Furthermore, for the same set of patients, the microcalcification activity data 205 (μCA_{Te}) should also be obtained so that the fitted model's error may be quantified.

### Step 4—Compute 211 the Error Estimate(s)

Having generated a set of estimates/predictions of microcalcification activity (μCA_{Est}) for the test data, they can be compared to the true microcalcification activity values (μCA_{Te}). The difference between each of these sets of data provides the error distribution. If the errors are normally distributed, a linear correlation of μCA_{Est} and μCA_{Te} is a very simple way to assess the model performance. The error distribution can also be simply used to assess the accuracy (ideally centred about zero) and precision of the model (ideally small in variance/range/spread). Other useful information can be generated from the error distribution, such as investigating the relationships between the errors and predictor(s) values or fitted values: useful in assessing the sensitivity of the model to the inputs/outputs.

The output of the model may also be converted to discrete/nominal classifications, which provides other ways in which the error estimates may be tested (e.g., sensitivity and specificity). In the case of binary classifications, this would be done through the measurement of true positives, false positives, true negatives, and false negatives. It would require a cut-off value to classify elevated microcalcification activity. This could be established for a set of control patients (with no suspected cardiovascular disease) and/or thresholding tissue to background ratio (e.g., above unity, the relative background value).

Step 4(a)—Check 213 the Error Estimate to Assess the Suitability of the Model

If the error estimates meet a set of desired criteria, such as a required accuracy, precision, and sensitivity to input data, then model is considered fit for purpose, and used to predict microcalcification activity.

### Preferred Embodiment

The following presents an illustration of a particular embodiment of the invention for better understanding the nature of the invention. A purely image-based example with parametric model generation is the coronary vasculature. Here the general patient data is obtained from intravascular optical coherence tomography (OCT) imaging and coronary computed tomography angiography (CCTA) imaging data. The microcalcification activity data is obtained from ¹⁸F—NaF PET imaging, following a registration step with the (contrast enhanced) CCTA imaging data (aligning both image spaces and the objects within them). The general patient data and microcalcification activity measurements are sampled in different regions of the coronary vasculature: the major coronary artery segments described by a commonly used coronary artery segment map. Following the data collection stage, a model (parametric) is fit and tested. All methods and results detailed in Table A below.

All training and testing data is collected at the same stage in this example.

In the case of using the system to determine microcalcification activity from coronary CT angiography (CCTA) data, the system receives raw CCTA image data and then determines the quality of these image data by accessing the associated image metadata; for instance, the slice thickness and pixel size of the CTCA acquisition must be above a specific threshold. If the quality control checks are passed, the system sorts the CCTA data for use in an AI training dataset. Where applicable, the system also compares morphology and plaque features identifiable on CCTA with corresponding features visible on invasive imaging (e.g., OCT) from the same patient. This is to verify that the identifiable features on CCTA spatially correlate with those identifiable on OCT. After quality control, data such as anatomical metrics and image-based distributions of pixel density, are extracted from the AI-derived geometry for use in the regression training model. If the quality control checks are not passed, the system returns an error stating that the raw CCTA data cannot be used for training data or further purposes with the system.

Step 1(i)—General Patient Data 203

These data sets are considered are considered as the independent variables (inputs or predictors) of the model, and are labelled as A, B, C, D and so on. Multiple measurements of each input are taken. In the case of three inputs (i.e., A, B and C), each patient would have multiple measurements for each of A, B and C obtained at multiple locations. For example, in the coronary vasculature, the example inputs may include artery tortuosity (Tort); low shear area (LSA) and plaque free wall (PFW). They would be measured in each coronary artery vessel segment within the vasculature spanning the spatial domain imaged by (and common to) all three imaging modalities.

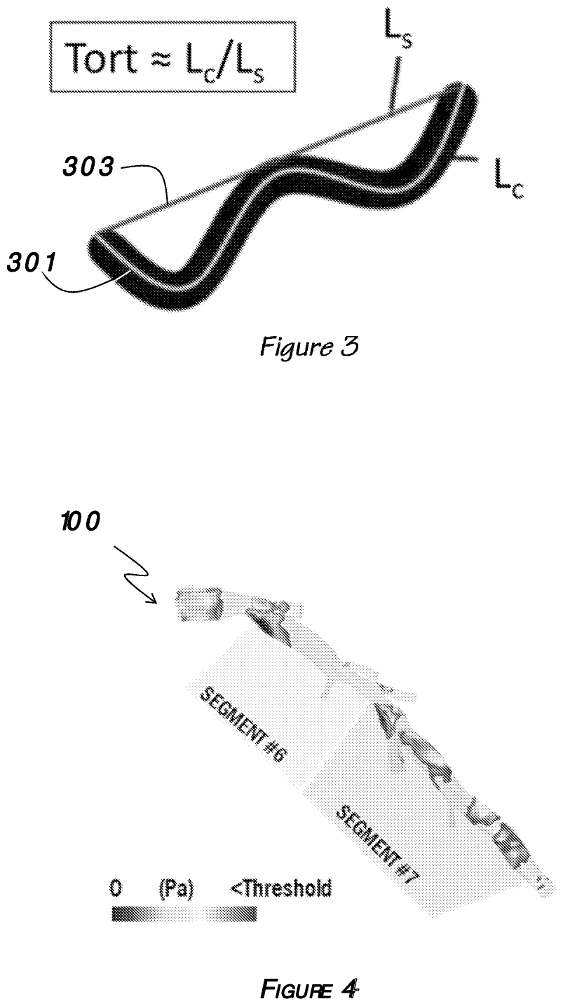

Tortuosity (Tort): Tortuosity of the vessel lumen centreline 301 (FIG. 3) measured from CCTA. Diseased vessels tend to have more tortuosity. The centreline of a vessel is typically constructed from the wall boundary distance field, or by using ray casting to compute wall distances, and may be reliably constructed using a number of centreline algorithms. Before this process, the vessel boundary must be defined, and here this was done through image segmentation: in this process the objects on images are thresholded (masked) within the region of interest between Hounsfield units/pixel-levels that are inclusive of the target object (vessel lumen) and exclusive of other, surrounding objects. Once the centreline data is extracted, the centreline tortuosity is simply measured here as the ratio of the total length, L_{C} 301, along the centreline segment (i.e., the sum of distances between consecutive points), divided by the shortest distance, L_{S} 303, (straight-line) between the end points of the centreline that bounds the region of interest where the patient-specific measurements are being obtained as shown in FIG. 3.

Low Shear Area (LSA): The percentage (proportion) of the vessel (segment) lumen surface area that has a wall shear stress value below a specific threshold (0.4 Pa used in this example, but 1 Pa is also typically used to describe low shear stress in the arterial system). This threshold is related to the near-wall stagnation of blood flow and increased monocyte wall adhesion. Low wall shear stress has been associated with the initiation and progression of atherosclerosis. Similar to the centreline reconstruction, it is computed from a domain defined by a vessel lumen boundary using computational fluid dynamics. For the current data set, this could be performed using either the vessel boundary segmented from the CCTA images or the OCT images. Choosing the OCT images (due to their superior pixel-resolution), the OCT vessel lumen boundary is registered to the CCTA image space, giving the OCT vessel boundary curvature, and allowing the OCT measurements to have spatial correspondence/alignment with the vessel's segments defined by the coronary segment map, from which each measurement region is defined. Below is an image showing the low shear area (in Pascals) identified on the surface an OCT-derived geometry after registration to the CCTA image space (see FIG. 4).

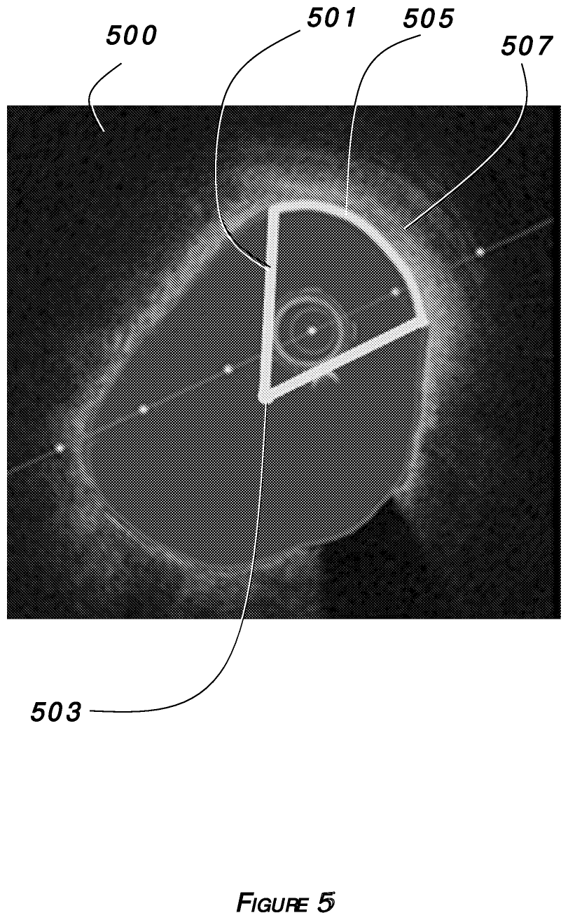

Plaque Free Wall (PFW): The plaque free wall is an OCT measurement that is taken on an OCT image e.g., image 500 of FIG. 5, as the angle 501 about the lumen centre 503 in which the vessel intima 505 and media 507 are healthy in regions where the artery wall is clearly visible and not obstructed by plaque features attenuating the OCT signal (it is inversely related to the presence of disease). The angular measurement method also allows the PFW to be mapped to the vessel boundary, such that is may also be represented as the percentage of a vessel segments lumen surface area. FIG. 5 is shown as an example of a PFW arc angle 501 overlayed on an OCT image 500.

Step 1(ii)—Microcalcification Activity Data 205

The dependent variables of the model (predicted variable/output) are the measurement of the microcalcification activity, on the ¹⁸F—NaF PET imaging, assessed for each vessel segment in the common image space. Such that each segment also has a corresponding general patient data measurement from each of the three categories, Tort, LSA and PFW, described above.

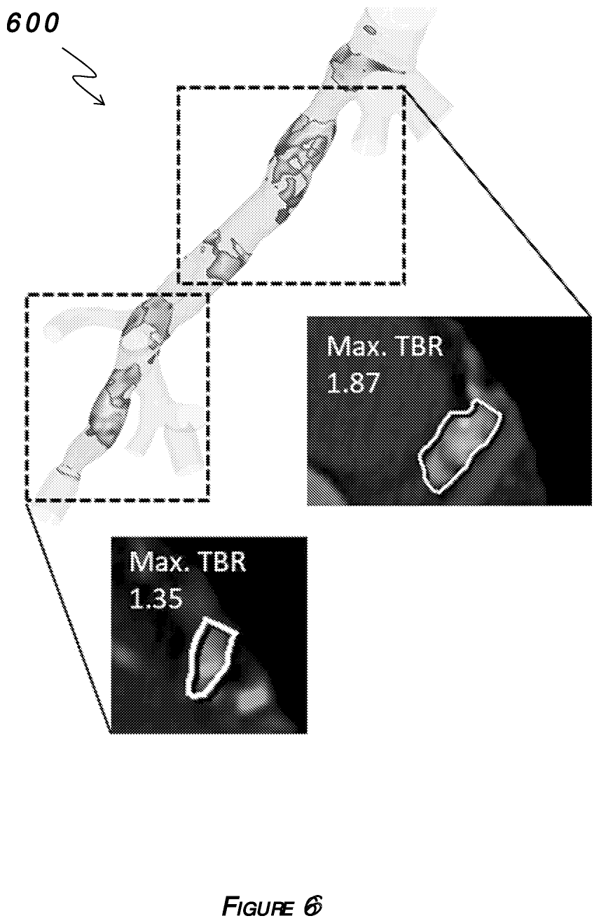

FIG. 6 shows an example image 600 of ¹⁸F—NaF PET segment measurements where the LSA surface area data is also displayed; both data-sets are obtained for the same anatomic regions.

The microcalcification activity is measured as the maximum of the tissue-to-background ratio (TBR) in each segment: the maximum standardised uptake value in the segment is normalised by the blood pool activity. Where the blood pool activity is measured as the mean standardised uptake value in the right atrium. The regions of interest in which the TBR is measured on the PET images include the coronary artery wall (as the microcalcification activity occurs in the wall, rather than the lumen).

### Step 2—Fit the Multivariate Function/Model

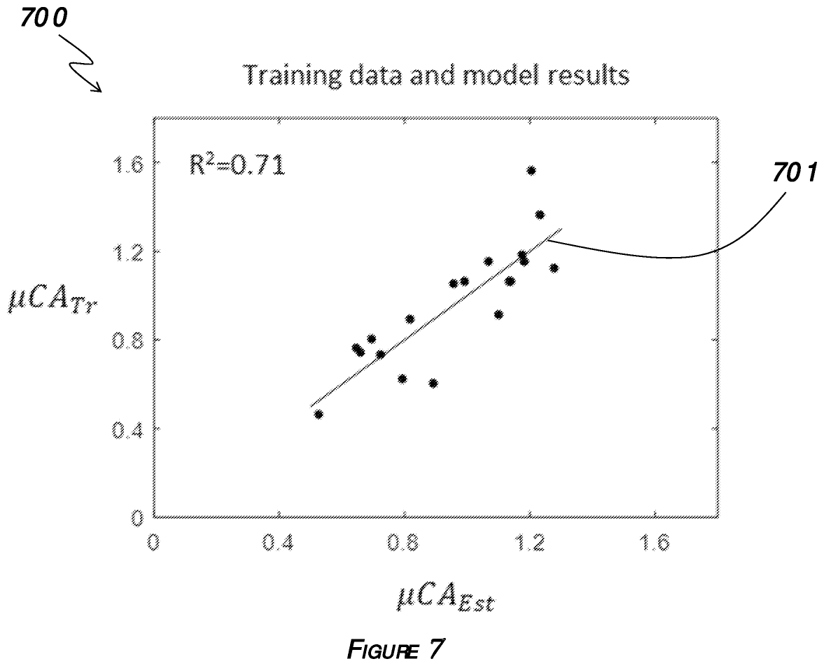

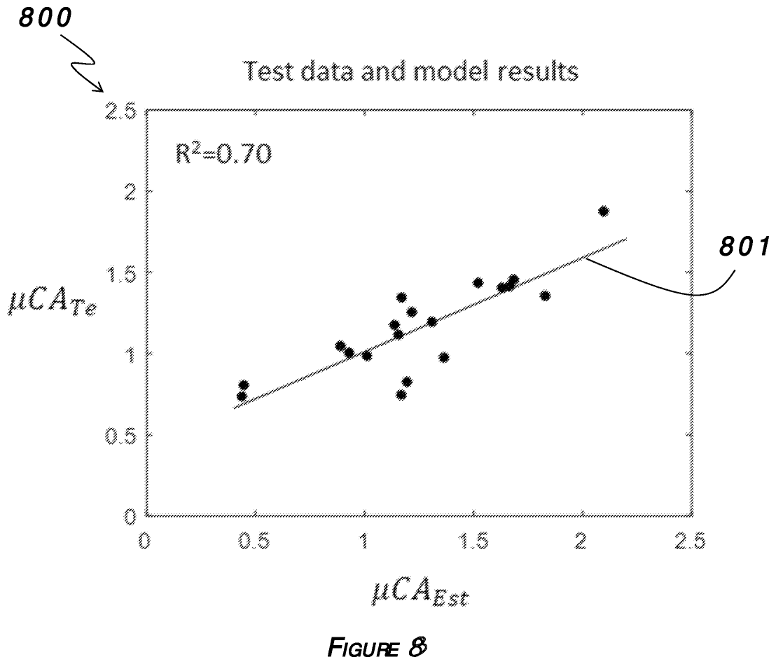

Here, half of the collected data is used for training the model. The multivariate model is assumed to have the following combination of power law equations, often used to represent allometric relationships:

$$μ C {A}_{E s t} = {a ( Tort )}^{b} + {c ( LSA )}^{d} + {e ( PFW )}^{f} ,$$

where a, b, c, d, e, and fare the coefficients of the model that are determined during model fitting and μCA_{Est} is the value of the model estimate of microcalcification activity (the maximum segment TBR value). In this example, to determine the coefficients, the sum of the square error (E) is minimised following the Nelder-Mead simplex algorithm:

$$ε = \sum {( μ {CA}_{T r} - μ {CA}_{Est} )}^{2} ,$$

where μCA_{Tr} is the vector/array containing all the measured values of maximum segment TBR in the set of training data and μCA_{Est} is the vector/array of estimate for a given set of coefficients during optimisation. Once the coefficients are generated, correlation analysis is performed to assess how well the model fits the training data. This correlation is expected to be high, considering that the model was optimised to fit this data. For a first order polynomial, the fit result 701 is shown in FIG. 7.

### Step 3—Evaluate the Multivariate Model, Compute of the Error Estimate(s) and Assess the Suitability of the Model

Having determined the coefficients of the model, it is evaluated for the remainder of the general patient data (data that was not in the training data used to fit the model). In contrast to training data, the first order polynomial fit 801 of FIG. 8 for this data is slightly weaker but still significant (R²Tᵣ=0.71; R²Tₑ=0.70; p-value Tₑ<0.0001).

Furthermore, the distribution of the error, obtained from Ef=μCA_{Est}−μCA_{Te}, was normally distributed with a mean of value of 0.099 and a standard deviation of 0.24. Provided that the mean value of μCA_{Te} is 1.16, the model tended to over predict microcalcification activity by ˜9% with moderate precision. Furthermore, negative association (Spearman's Rho) was found between the PFW values and the error values (Rho=−0.73, p-value<0.001), suggesting that the current model is more prone to under predict the microcalcification activity in healthier vessel segments, as measured using the PFW metric.

Considering this information, the model may be of use for patients presenting with CCTA and OCT imaging of the coronary vasculature, but could benefit from excluding PFW and/or using another metric in its place.

Additional objectives, advantages and novel features will be set forth in the description which follows or will become apparent to those skilled in the art upon examination of the drawings and the ensuing detailed description of several non-limiting embodiments which follows.

### Examples

In an example of the invention different categorical data is obtained in accordance with Table 1.

TABLE 1
Categorical measurements obtained from different sources: intravascular
optical coherence tomography (OCT), computational fluid dynamic
simulations (CFD), computed tomography (CT) and blood test

| Column 1 | Column 2 | | Column 4 | Column 5 |
|---|---|---|---|---|
| OCT | OCT | Column 3 | OCT + CT | Blood Test |
| (plaque | (health | CFD | (geometric | (non-image based |
| measurements) | measurements) | (biomechanics) | measurements | measurements) |
| Avg. Lipid | Avg. | LSA: Area of | Avg. | Haematocrit [%] |
| Arc [°] | Plaque-free | Low WSS [%] | Circumference | |
| Avg. Calcium | Wall Arc [°] | HSA: Area of | [mm] (OCT) | |
| Arc [°] | | High WSS [%] | Avg. | |
| Avg. Bright | | Avg. WSS [Pa] | Eccentricity | |
| Spots | | | (OCT) | |
| | | | Left Ventricular | |
| | | | Muscle Mass | |
| | | | [g] (CT) | |

The following results show linear regressions between the measured maximum ¹⁸F—NaF PET uptake (the maximum target-to-background ratio (TBR)) and the estimated microcalcification activity, P, computed using equation (1) for P₁ above.

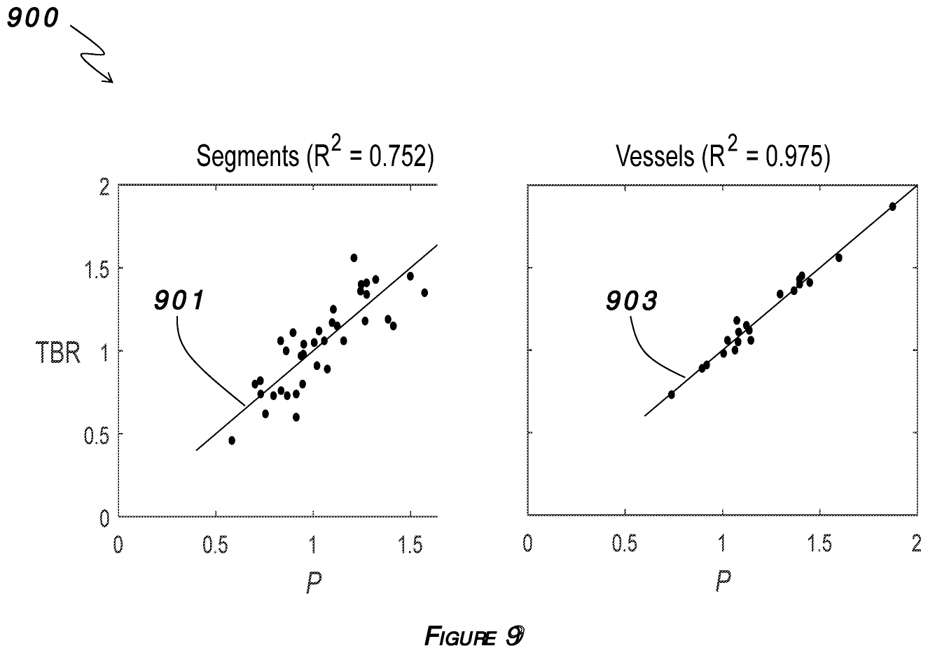

In FIG. 9 all measurements in Table 1 are used for fitting, with comparisons performed at the coronary segment and coronary vessel (i.e., whole OCT-pullback) measurement scales. Note, the size of each segment used to obtain the measurements varies, and the maximum TBR at the vessel scale is the maximum TBR of all segments spanning a vessel.

In FIG. 9, the fits 901 and 903 demonstrate strong linear relationships between the models and the data. At the vessel measurement scale, the number of degrees of freedom of the fitted model (23 coefficients) exceeds the number of data points (20) used to optimise/fit the model coefficients. Therefore the present example model is almost certainly overfitting the data: the resulting model may fail to fit additional data or predict future observations reliably.

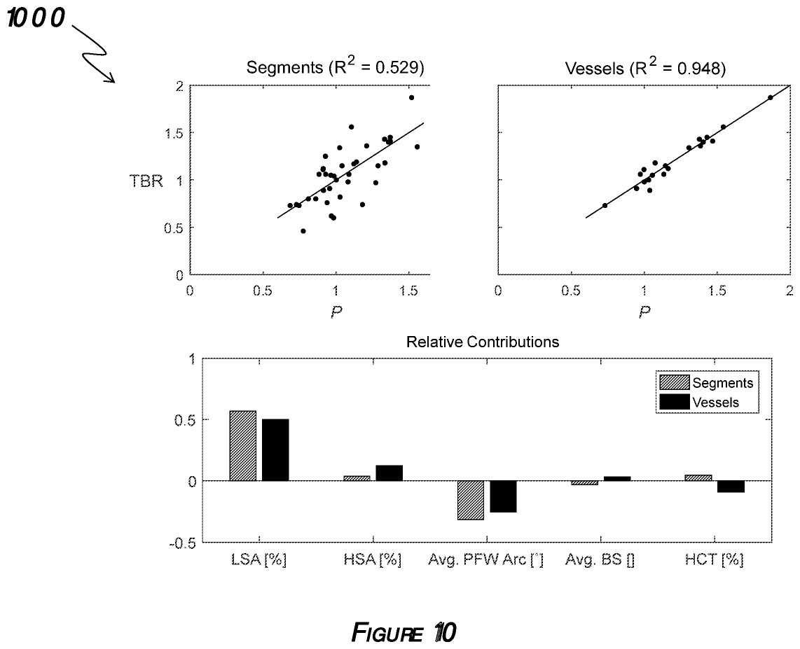

In FIG. 10, it can be seen by reducing the number of categorical measurements used, while ignoring geometric measures, the models still performs well at the Vessel measurement scale, and there is less risk of overfitting the data. However, the performance at the Segment measurement scale is not maintained. In this group of variables, the normalised area of low WSS (<0.4 Pa) (LSA [%]) and the average plaque-free wall arc (Avg. PFW Arc [° ]) have the greatest (average) contributions to the approximation of microcalcification activity.

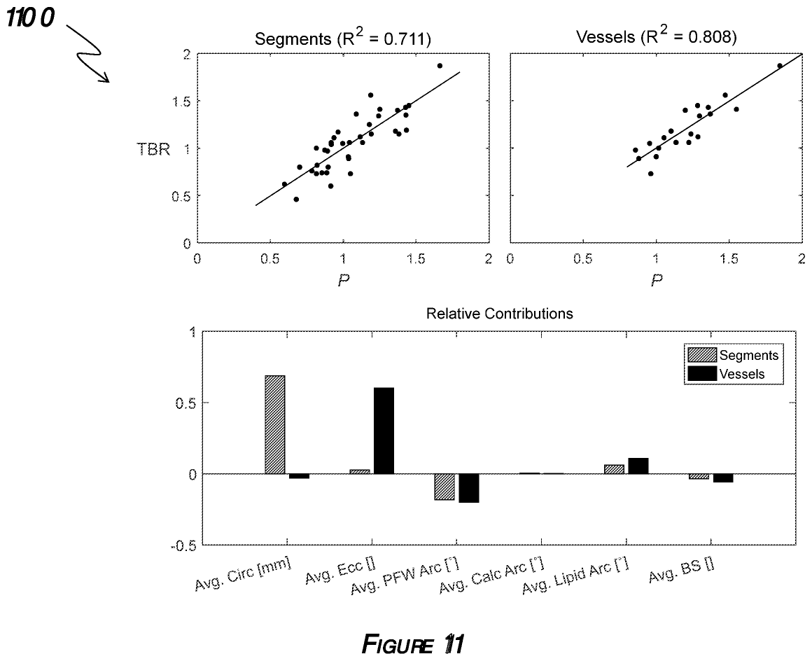

Furthermore, by using only OCT-derived metrics models can be generate which perform well at both measurement scales (FIG. 11). Interestingly, when fit at the different measurement scales, the resulting models differ in the weights they apply to the different geometric measurements: circumference and eccentricity. The optimisation process is dependent on the initial conditions and does not necessarily find the global minima. However, variation in the models is not unexpected. Localised geometry measurements are indicative of segment location and vary throughout the coronary vasculature, along with the occurrence of plaque phenotypes. Proximal vessel segments are typically larger than distal segments and high-risk plaque tend to form in the proximal vasculature.

The relationship between these geometric variables and the TBR, and LSA measurements in Table 2, shows that, in isolation, both circumference and eccentricity are positively related to TBR and LSA. However, the correlation coefficient for eccentricity is weak, while circumference presents slightly stronger relationships. It is apparent that the strength of their individual correlations do not reflect their contributions to the multivariate models, where the relationship between all variables influences the outcome of the model(s).

TABLE 2
Low-dimensional power-law model correlations (linear regression).
Note, LSA is not normally distributed and relationships with it
may be better repressed by rank correlations

| | Max. TBR | Max. TBR | LSA | LSA |
|---|---|---|---|---|
| | (Segments) | (Vessels) | (Segments) | (Vessels) |
| Model | R | R | R | R |
| a(Circ)^{b} | 0.518 | 0.383 | 0.341 | 0.444 |
| a(Ecc)^{b} | 0.255 | 0.298 | 0.045 | 0.176 |
| ${a ( \frac{E c c \times C i r c}{H C T \times {LVM}^{\frac{3}{4}}} )}^{b}$ | 0.610 | 0.522 | 0.663 | 0.789 |
| ${a ( \frac{C i r c}{H C T \times {LVM}^{\frac{3}{4}}} )}^{b}$ | 0.601 | 0.496 | 0.750 | 0.860 |
| a(LSA)^{b} | 0.521 | 0.636 | 1.000 | 1.000 |

In Table 2 there are additional, simple, models for approximating LSA, which incorporate factors influencing blood supply (LVM^{3/4}) and viscosity (HCT). These models provide improved approximations of microcalcification activity, TBR, compared to the geometric measurements alone, and are competitive with the CFD measurement of LSA. The inclusion of these simple models in the previous six-parameter OCT-derived multivariate models (FIG. 11) improves the correlation coefficients (R²_{Seg}: 0.71 to 0.72; R²_{Ves}: 0.81 to 0.90), however, no more than including HCT and LVM^{3/4} as independent parameters with power-law scaling coefficients (R²_{Seg}=0.75; R²_{Ves}=0.88).

At present, it can be concluded that a simple multivariate function, such as P₁, in Equation (1) described above, can be tuned to provide a model that fits the data well, while being dependent on an easily acquirable subset of categorical measurements. However, other alternatives are available, including those where the form of the multivariate function is not defined prior to fitting.

This method is limited by the amount of consistent data available (applied only to the segment data here). For the current data set it fit the training data well, similar to the function P₁ in FIGS. 9 to 11, however it failed to predict test data.

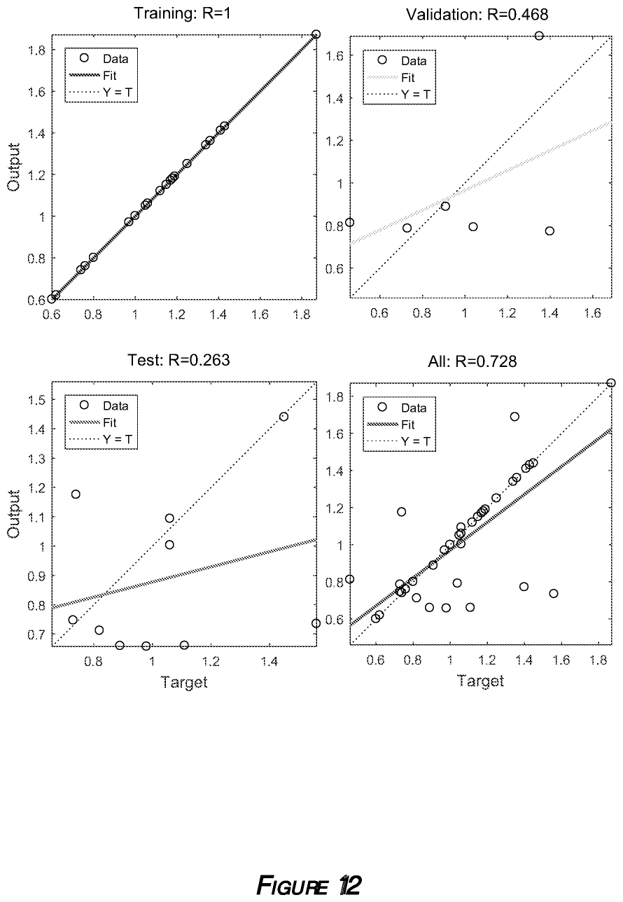

A particular alternative is a machine learning method that performs data-driven fitting (non-parametric), where the equation for P is generated. A two-layer feed-forward network with sigmoid hidden neurons and linear output neurons is an example implementation. This model that can fit multi-dimensional mapping problems arbitrarily well, given that the data is consistent and the model has enough neurons in its hidden layer. The network is implemented using the Levenberg-Marquardt backpropagation algorithm and the default value of ten neurons in the hidden layer. However, it is not immune from over fitting. The performance of the model was tested for different ratios of training, validation, and test data. In some instances, shown in FIG. 12 which provide specific examples of AI-based fitting, the training data is fit perfectly, however it is clear the model is overfit and does not work well with future data.

This issue also occurs when fitting high dimensional models (>3 arguments) of P₁ to training and test sets, such that the true predictive power of the models in FIGS. 10 and 11 is unknown without further data. However, some analysis has been performed using low dimensional models (Table 3 and Table 4 below). These models show promise, but would benefit from further scrutiny on larger data sets. The LSA and PFW model displays consistency across all training/testing dataset ratios. All models perform well when provided a higher proportion of data for training.

TABLE 3
Assessment of predictive power of low dimensional models at the vessel scale

| | Max. TBR | Max. TBR | Max. TBR |
|---|---|---|---|
| | Rₜᵣₐᵢₙ, Rₜₑₛₜ | Rₜᵣₐᵢₙ, Rₜₑₛₜ | Rₜᵣₐᵢₙ, Rₜₑₛₜ |
| Model | Training set: 35% | Training set: 50% | Training set: 65% |
| a(LSA)^{b} + c(PFW)^{d} | 0.846, 0.754 | 0.808, 0.730 | 0.886, 0.815 |
| a(Circ)^{b} + c(Ecc)^{d} + | 0.998, 0.484 | 0.904, 0.627 | 0.863, 0.821 |
| e(PFW)^{f} | | | |
| ${a ( \frac{E c c \times C i r c}{H C T \times {LVM}^{\frac{3}{4}}} )}^{b} + {c ( P F W )}^{d}$ | 0.973, 0.422 | 0.706, 0.709 | 0.827, 0.817 |

TABLE 4
Assessment of predictive power of low dimensional models at the segment scale

| | Max. TBR | Max. TBR | Max. TBR |
|---|---|---|---|
| | Rtrain, Rtest | Rtrain, Rtest | Rtrain, Rtest |
| Model | Training set: 35% | Training set: 50% | Training set: 65% |
| a(LSA)^{b} + c(PFW)^{d} | 0.636, 0.635 | 0.628, 0.628 | 0.736, 0.601 |
| a(Circ)^{b} + c(Ecc)^{d} + | 0.858, 0.243 | 0.782, 0.503 | 0.767, 0.727 |
| e(PFW)^{f} | | | |
| ${a ( \frac{Ecc \times Circ}{H C T \times {LVM}^{\frac{3}{4}}} )}^{b} + {c ( P F W )}^{d}$ | 0.571, 0.565 | 0.611, 0.649 | 0.751, 0.734 |

The presented methods show that measurable microcalcification activity detected using ¹⁸F—NaF PET is predictable using measurements of the local hemodynamic environment, as well as the existence/absence of coronary plaque, and related metrics. Both the absence of plaque-free wall, a general marker for disease, and presence of low endothelial shear stress areas were integral to the multivariate models discussed.

### Implementation Example—Hardware Overview

According to one embodiment, the techniques described herein are implemented by at least one computing device. The techniques may be implemented in whole or in part using a combination of at least one server computer and/or other computing devices that are coupled using a network, such as a packet data network. The computing devices may be hard-wired to perform the techniques, or may include digital electronic devices such as at least one application-specific integrated circuit (ASIC) or field programmable gate array (FPGA) that is persistently programmed to perform the techniques, or may include at least one general purpose hardware processor programmed to perform the techniques pursuant to program instructions in firmware, memory, other storage, or a combination. Such computing devices may also combine custom hard-wired logic, ASICs, or FPGAs with custom programming to accomplish the described techniques. The computing devices may be server computers, workstations, personal computers, portable computer systems, handheld devices, mobile computing devices, wearable devices, body mounted or implantable devices, smartphones, smart appliances, internetworking devices, autonomous or semi-autonomous devices such as robots or unmanned ground or aerial vehicles, any other electronic device that incorporates hard-wired and/or program logic to implement the described techniques, one or more virtual computing machines or instances in a data centre, and/or a network of server computers and/or personal computers.

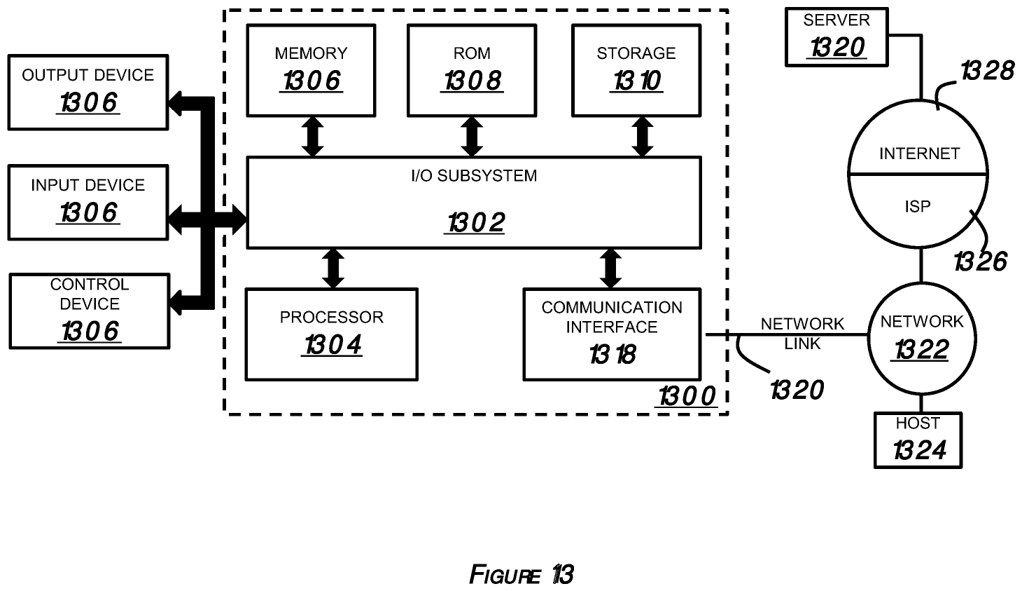

FIG. 13 is a block diagram that illustrates an example computer system with which an embodiment of system 100 described above may be implemented. In the example of FIG. 13, a computer system 1300 and instructions for implementing the disclosed technologies in hardware, software, or a combination of hardware and software, are represented schematically, for example as boxes and circles, at the same level of detail that is commonly used by persons of ordinary skill in the art to which this disclosure pertains for communicating about computer architecture and computer systems implementations.

Computer system 1300 includes an input/output (I/O) subsystem 1302 which may include a bus and/or other communication mechanism(s) for communicating information and/or instructions between the components of the computer system 1300 over electronic signal paths. The I/O subsystem 1302 may include an I/O controller, a memory controller and at least one I/O port. The electronic signal paths are represented schematically in the drawings, for example as lines, unidirectional arrows, or bidirectional arrows.

At least one or more hardware processor(s) 1304 is coupled to I/O subsystem 1302 for processing information and instructions. Hardware processor 1304 may include, for example, a general-purpose microprocessor or microcontroller and/or a special-purpose microprocessor such as an embedded system or a graphics processing unit (GPU) or a digital signal processor or ARM processor. Processor 1304 may comprise an integrated arithmetic logic unit (ALU) or may be coupled to a separate ALU. One or more of hardware processors 1304 may be implemented as dedicated image processing means for segmenting, annotating and otherwise analysing patient image data. Alternatively, image processing means functions may be shared among each of the hardware processors 1304.

Computer system 1300 includes one or more units of memory 1306, such as a main memory, which is coupled to I/O subsystem 1302 for electronically digitally storing data and instructions to be executed by processor 1304. Memory 1306 is also used for storing patient image data and training data for retrieval by processors 1304. Memory 1306 may include volatile memory such as various forms of random-access memory (RAM) or other dynamic storage device. Memory 1306 also may be used for storing temporary variables or other intermediate information during execution of instructions to be executed by processor 1304. Such instructions, when stored in non-transitory computer-readable storage media accessible to processor 1304, can render computer system 1300 into a special-purpose machine that is customized to perform the operations specified in the instructions.

Computer system 1300 further includes non-volatile memory such as read only memory (ROM) 1308 or other static storage device coupled to I/O subsystem 1302 for storing information and instructions for processor 1304. The ROM 1308 may include various forms of programmable ROM (PROM) such as erasable PROM (EPROM) or electrically erasable PROM (EEPROM). A unit of persistent storage 1310 may include various forms of non-volatile RAM (NVRAM), such as FLASH memory, or solid-state storage, magnetic disk, or optical disk such as CD-ROM or DVD-ROM, and may be coupled to I/O subsystem 1302 for storing information and instructions. Storage 1310 is an example of a non-transitory computer-readable medium that may be used to store instructions and data which when executed by the processor 1304 cause performing computer-implemented methods to execute the techniques herein.

The instructions in memory 1306, ROM 1308 or storage 1310 may comprise one or more sets of instructions that are organized as modules, methods, objects, functions, routines, or calls. The instructions may be organized as one or more computer programs, operating system services, or application programs including mobile apps. The instructions may comprise an operating system and/or system software; one or more libraries to support multimedia, programming or other functions; data protocol instructions or stacks to implement TCP/IP, HTTP or other communication protocols; file format processing instructions to parse or render files coded using HTML, XML, JPEG, MPEG or PNG; user interface instructions to render or interpret commands for a graphical user interface (GUI), command-line interface or text user interface; application software such as an office suite, internet access applications, design and manufacturing applications, graphics applications, audio applications, software engineering applications, educational applications, games or miscellaneous applications. The instructions may implement a web server, web application server or web client. The instructions may be organized as a presentation layer, application layer and data storage layer such as a relational database system using structured query language (SQL) or no SQL, an object store, a graph database, a flat file system or other data storage.

Computer system 1300 may be coupled via I/O subsystem 1302 to at least one output device 1312. In one embodiment, output device 1312 is a digital computer display. Examples of a display that may be used in various embodiments include a touch screen display or a light-emitting diode (LED) display or a liquid crystal display (LCD) or an e-paper display. Computer system 1300 may include other type(s) of output devices 1312, alternatively or in addition to a display device. Examples of other output devices 1312 include printers, ticket printers, plotters, projectors, sound cards or video cards, speakers, buzzers or piezoelectric devices or other audible devices, lamps or LED or LCD indicators, haptic devices, actuators, or servos.

At least one input device 1314 is coupled to I/O subsystem 1302 for communicating signals, data, command selections or gestures to processor 1304. Examples of input devices 1314 include touch screens, microphones, still and video digital cameras, alphanumeric and other keys, keypads, keyboards, graphics tablets, image scanners, joysticks, clocks, switches, buttons, dials, slides, and/or various types of sensors such as force sensors, motion sensors, heat sensors, accelerometers, gyroscopes, and inertial measurement unit (IMU) sensors and/or various types of transceivers such as wireless, such as cellular or Wi-Fi, radio frequency (RF) or infrared (IR) transceivers and Global Positioning System (GPS) transceivers.

Another type of input device is a control device 1316, which may perform cursor control or other automated control functions such as navigation in a graphical interface on a display screen, alternatively or in addition to input functions. Control device 1316 may be a touchpad, a mouse, a trackball, or cursor direction keys for communicating direction information and command selections to processor 1304 and for controlling cursor movement on display 1312. The input device may have at least two degrees of freedom in two axes, a first axis (e.g., x) and a second axis (e.g., y), that allows the device to specify positions in a plane. Another type of input device is a wired, wireless, or optical control device such as a joystick, wand, console, steering wheel, pedal, gearshift mechanism or other type of control device. An input device 1314 may include a combination of multiple different input devices, such as a video camera and a depth sensor.

In another embodiment, computer system 1300 may comprise an internet of things (IoT) device in which one or more of the output device 1312, input device 1314, and control device 1316 are omitted. Or, in such an embodiment, the input device 1314 may comprise one or more cameras, motion detectors, thermometers, microphones, seismic detectors, other sensors or detectors, measurement devices or encoders and the output device 1312 may comprise a special-purpose display such as a single-line LED or LCD display, one or more indicators, a display panel, a meter, a valve, a solenoid, an actuator or a servo.

When computer system 1300 is a mobile computing device, input device 1314 may comprise a global positioning system (GPS) receiver coupled to a GPS module that is capable of triangulating to a plurality of GPS satellites, determining and generating geo-location or position data such as latitude-longitude values for a geophysical location of the computer system 1300. Output device 1312 may include hardware, software, firmware, and interfaces for generating position reporting packets, notifications, pulse or heartbeat signals, or other recurring data transmissions that specify a position of the computer system 1300, alone or in combination with other application-specific data, directed toward host 1324 or server 1330.

Computer system 1300 may implement the techniques described herein using customized hard-wired logic, at least one ASIC or FPGA, firmware and/or program instructions or logic which when loaded and used or executed in combination with the computer system causes or programs the computer system to operate as a special-purpose machine. According to one embodiment, the techniques herein are performed by computer system 1300 in response to processor 1304 executing at least one sequence of at least one instruction contained in main memory 1306. Such instructions may be read into main memory 1306 from another storage medium, such as storage 1310. Execution of the sequences of instructions contained in main memory 1306 causes processor 1304 to perform the process steps described herein. In alternative embodiments, hard-wired circuitry may be used in place of or in combination with software instructions.

The term “storage media” as used herein refers to any non-transitory media that store data and/or instructions that cause a machine to operation in a specific fashion. Such storage media may comprise non-volatile media and/or volatile media. Non-volatile media includes, for example, optical or magnetic disks, such as storage 1310. Volatile media includes dynamic memory, such as memory 1306. Common forms of storage media include, for example, a hard disk, solid state drive, flash drive, magnetic data storage medium, any optical or physical data storage medium, memory chip, or the like.

Storage media is distinct from but may be used in conjunction with transmission media. Transmission media participates in transferring information between storage media. For example, transmission media includes coaxial cables, copper wire and fibre optics, including the wires that comprise a bus of I/O subsystem 1302. Transmission media can also take the form of acoustic or light waves, such as those generated during radio-wave and infra-red data communications.

Various forms of media may be involved in carrying at least one sequence of at least one instruction to processor 1304 for execution. For example, the instructions may initially be carried on a magnetic disk or solid-state drive of a remote computer. The remote computer can load the instructions into its dynamic memory and send the instructions over a communication link such as a fibre optic or coaxial cable or telephone line using a modem. A modem or router local to computer system 1300 can receive the data on the communication link and convert the data to a format that can be read by computer system 1300. For instance, a receiver such as a radio frequency antenna or an infrared detector can receive the data carried in a wireless or optical signal and appropriate circuitry can provide the data to I/O subsystem 1302 such as place the data on a bus. I/O subsystem 1302 carries the data to memory 1306, from which processor 1304 retrieves and executes the instructions. The instructions received by memory 1306 may optionally be stored on storage 1310 either before or after execution by processor 1304.

Computer system 1300 also includes a communication interface 1318 coupled to bus 1302. Communication interface 1318 provides a two-way data communication coupling to network link(s) 1320 that are directly or indirectly connected to at least one communication networks, such as a network 1322 or a public or private cloud on the Internet. For example, communication interface 1318 may be an Ethernet networking interface, integrated-services digital network (ISDN) card, cable modem, satellite modem, or a modem to provide a data communication connection to a corresponding type of communications line, for example an Ethernet cable or a metal cable of any kind or a fibre-optic line or a telephone line. Network 1322 broadly represents a local area network (LAN), wide-area network (WAN), campus network, internetwork, or any combination thereof. Communication interface 1318 may comprise a LAN card to provide a data communication connection to a compatible LAN, or a cellular radiotelephone interface that is wired to send or receive cellular data according to cellular radiotelephone wireless networking standards, or a satellite radio interface that is wired to send or receive digital data according to satellite wireless networking standards. In any such implementation, communication interface 1318 sends and receives electrical, electromagnetic, or optical signals over signal paths that carry digital data streams representing various types of information.

Network link 1320 typically provides electrical, electromagnetic, or optical data communication directly or through at least one network to other data devices, using, for example, satellite, cellular, Wi-Fi, or BLUETOOTH technology. For example, network link 1320 may provide a connection through a network 1322 to a host computer 1324.

Furthermore, network link 1320 may provide a connection through network 1322 or to other computing devices via internetworking devices and/or computers that are operated by an Internet Service Provider (ISP) 1326. ISP 1326 provides data communication services through a world-wide packet data communication network represented as internet 1328. A server computer 1330 may be coupled to internet 1328. Server 1330 broadly represents any computer, data centre, virtual machine, or virtual computing instance with or without a hypervisor, or computer executing a containerized program system such as DOCKER or KUBERNETES. Server 1330 may represent an electronic digital service that is implemented using more than one computer or instance and that is accessed and used by transmitting web services requests, uniform resource locator (URL) strings with parameters in HTTP payloads, API calls, app services calls, or other service calls. Computer system 1300 and server 1330 may form elements of a distributed computing system that includes other computers, a processing cluster, server farm or other organization of computers that cooperate to perform tasks or execute applications or services. Server 1330 may comprise one or more sets of instructions that are organized as modules, methods, objects, functions, routines, or calls. The instructions may be organized as one or more computer programs, operating system services, or application programs including mobile apps. The instructions may comprise an operating system and/or system software; one or more libraries to support multimedia, programming or other functions; data protocol instructions or stacks to implement TCP/IP, HTTP or other communication protocols; file format processing instructions to parse or render files coded using HTML, XML, JPEG, MPEG or PNG; user interface instructions to render or interpret commands for a graphical user interface (GUI), command-line interface or text user interface; application software such as an office suite, internet access applications, design and manufacturing applications, graphics applications, audio applications, software engineering applications, educational applications, games or miscellaneous applications. Server 1330 may comprise a web application server that hosts a presentation layer, application layer and data storage layer such as a relational database system using structured query language (SQL) or no SQL, an object store, a graph database, a flat file system or other data storage.

Computer system 1300 can send messages and receive data and instructions, including program code, through the network(s), network link 1320 and communication interface 1318. In the Internet example, a server 1330 might transmit a requested code for an application program through Internet 1328, ISP 1326, local network 1322 and communication interface 1318. The received code may be executed by processor 1304 as it is received, and/or stored in storage 1310, or other non-volatile storage for later execution.

The execution of instructions as described in this section may implement a process in the form of an instance of a computer program that is being executed, and consisting of program code and its current activity. Depending on the operating system (OS), a process may be made up of multiple threads of execution that execute instructions concurrently. In this context, a computer program is a passive collection of instructions, while a process may be the actual execution of those instructions. Several processes may be associated with the same program; for example, opening up several instances of the same program often means more than one process is being executed. Multitasking may be implemented to allow multiple processes to share processor 1304. While each processor 1304 or core of the processor executes a single task at a time, computer system 1300 may be programmed to implement multitasking to allow each processor to switch between tasks that are being executed without having to wait for each task to finish. In an embodiment, switches may be performed when tasks perform input/output operations, when a task indicates that it can be switched, or on hardware interrupts. Time-sharing may be implemented to allow fast response for interactive user applications by rapidly performing context switches to provide the appearance of concurrent execution of multiple processes simultaneously. In an embodiment, for security and reliability, an operating system may prevent direct communication between independent processes, providing strictly mediated and controlled inter-process communication functionality.

The term “cloud computing” is generally used herein to describe a computing model which enables on-demand access to a shared pool of computing resources, such as computer networks, servers, software applications, and services, and which allows for rapid provisioning and release of resources with minimal management effort or service provider interaction.

A cloud computing environment (sometimes referred to as a cloud environment, or a cloud) can be implemented in a variety of different ways to best suit different requirements. For example, in a public cloud environment, the underlying computing infrastructure is owned by an organization that makes its cloud services available to other organizations or to the general public. In contrast, a private cloud environment is generally intended solely for use by, or within, a single organization. A community cloud is intended to be shared by several organizations within a community; while a hybrid cloud comprises two or more types of cloud (e.g., private, community, or public) that are bound together by data and application portability.

Generally, a cloud computing model enables some of those responsibilities which previously may have been provided by an organization's own information technology department, to instead be delivered as service layers within a cloud environment, for use by consumers (either within or external to the organization, according to the cloud's public/private nature). Depending on the particular implementation, the precise definition of components or features provided by or within each cloud service layer can vary, but common examples include: Software as a Service (SaaS), in which consumers use software applications that are running upon a cloud infrastructure, while a SaaS provider manages or controls the underlying cloud infrastructure and applications. Platform as a Service (PaaS), in which consumers can use software programming languages and development tools supported by a PaaS provider to develop, deploy, and otherwise control their own applications, while the PaaS provider manages or controls other aspects of the cloud environment (i.e., everything below the run-time execution environment). Infrastructure as a Service (IaaS), in which consumers can deploy and run arbitrary software applications, and/or provision processing, storage, networks, and other fundamental computing resources, while an IaaS provider manages or controls the underlying physical cloud infrastructure (i.e., everything below the operating system layer). Database as a Service (DBaaS) in which consumers use a database server or Database Management System that is running upon a cloud infrastructure, while a DBaaS provider manages or controls the underlying cloud infrastructure, applications, and servers, including one or more database servers.

1. A method of predicting microcalcification activity in a vascular vessel comprising either an artery or a vein, comprising the steps of:
(a) measuring patient data comprising one or more of:
(i) the existence of and/or quantity of coronary plaques or visible markers of disease in a vascular tissue sample;
(ii) the existence of and/or quantity of healthy tissue in the vascular tissue sample;
(iii) one or more features that define an abnormal hemodynamic environment in a vessel;
(iv) one or more geometric features that are associated with vascular remodeling and which influence hemodynamics in a vessel, and/or
(v) one or more material properties that influence vascular hemodynamics; and
(b) calculating the microcalcification activity in the vessel as a function of the measurements taken in Step (a).

2. The method of claim 1, wherein the vascular tissue sample comprises a patient's vascular system.

3. A method of claim 1, wherein the measurements of Step (a) are associated with the of the radiotracer ¹⁸F-sodium fluoride (NaF).

4. A method of claim 1, wherein the measurements of Step (a) are derived from one or more patient image sources.

5. The method of claim 4 wherein the one or more patient image sources are selected from the group comprising one or more of:
computer tomography;
optical coherence tomography;
intravascular ultrasound;
x-ray angiography;
PET imaging.

6. The method of claim 4, wherein the measurements are obtained by segmenting and annotating the patient image date using image processing means.

7. The method of claim 6, wherein the measurements of the vessel tissue comprise one or more of:
tortuosity of the vessel lumen centerline;
the percentage of the vessel lumen surface area that has a wall shear stress value below a predetermined threshold; or
the plaque free wall of the vessel tissue.

8. The method of claim 7, wherein the microcalcification activity is measured as the maximum of the tissue-to-background ratio (TBR) in each segment of the vessel tissue.

9. The method of claim 1 wherein the measurements of Step (a) include biomechanical measurements selected from the group of one or more of:
blood pressure;
blood flow rate or localised hemodynamic characteristics; and
tissue stresses.

10. A method of claim 1, wherein the one or more geometric features correspond with atherosclerotic processes and or microcalcification activity.

11. A method of claim 1, wherein the one or more geometric features correspond to image-based diameter measurements in a vessel prone to calcification.

12-15. (canceled)

16. The method of claim 1, wherein the vessel is one or more of a coronary artery, carotid artery, cerebral artery, aorta, peripheral artery, or vein.

17. A method of providing information for predicting the uptake of ¹⁸F-NAF in vascular tissues of a patient, comprising:
using image processing means on patient image data, measuring vascular biomarkers indicative of the existence of and/or quantity of coronary plaques or visible markers of disease in the vascular tissue associated with cardiovascular disease progression; and
using a processor, calculating the microcalcification activity in the vascular tissue as a function of the measurements.

18. The method of claim 1, comprising measuring microcalcification activity in a coronary artery, carotid artery, cerebral artery, aorta, peripheral artery, or any vessel of interest, including veins.

19. (canceled)

20. The method of claim 1, wherein the patient data comprises biomarker data relating to one or more features of clinical interest selected from the group of:
lipid region;
superficial calcium;
deep calcium;
plaque free wall;
thrombus;
macrophages;
microchannels;
cholesterol crystals; or
thin cap fibro-atheroma in relation to one or more blood vessels of the patient.

21. The method of claim 1, wherein the patient data comprises one or more of image data selected from the group of:
OCT image data;
angiography image data;
computed tomography (CT) image data;
CT angiography image data.

22. The method of claim 1, further comprising estimating the in vivo material properties based on ratios of tissue stiffness.

23. The method of claim 1, further comprising determining one or more measures of vessel status selected from the group comprising:
endoluminal sheer stress;
plaque structural stress;
plaque feature analysis;
microcalcification activity;
virtual stenting;
vessel wall feature analysis;
thin cap measurement;
multimodal imaging; vessel branches;
fractional flow reserve;
rapid timeframes; and
VR virtualisation.

24. The method of claim 1, wherein the existence and/or quantity of vascular plaques is measured based on measuring geometric markers of disease from intravascular patient image data, said geometric markers being selected from one or more of
lipid;
calcium; and
macrophages in plaque detected in the vascular vessel.

25. (canceled)

26. A computer system comprising:
at least one processor;
at least one memory device storing patient data relating to:
(i) the existence of and/or quantity of coronary plaques or visible markers of disease in a vascular tissue sample; and/or
(ii) the existence of and/or quantity of healthy tissue in the vascular tissue sample; and/or
(iii) one or more features that define an abnormal hemodynamic environment in a vessel; and/or
(iv) one or more geometric features that are associated with vascular remodeling and which influence hemodynamics in a vessel, and/or
(v) one or more material properties that influence vascular hemodynamics; and wherein the at least one processor is configured for, using a trained machine learning model, regression model or predictive model, calculating the microcalcification activity in the vessel as a function of the patient data;
a prediction processor for accessing an AI-trained model of the patient data and predicting ¹⁸F—NaF uptake in vascular tissues of the patient.
